SEQUENCE LISTING (56 sequences)

SEQ ID NO: 1 - DNA, 20 bp, Artificial Sequence
  Sense primer for the Nanog gene
atgcctcacacggagactgt

SEQ ID NO: 2 - DNA, 20 bp, Artificial Sequence
  Antisense primer for the Nanog gene
cagggctgtcctgaataagc

SEQ ID NO: 3 - DNA, 21 bp, Artificial Sequence
  Sense primer for the Sox2 gene
gggggaatggaccttgtatag

SEQ ID NO: 4 - DNA, 20 bp, Artificial Sequence
  Antisense primer for the Sox2 gene
gcaaagctcctaccgtacca

SEQ ID NO: 5 - DNA, 23 bp, Artificial Sequence
  Sense primer for the oct4 gene
gcttcaagaacatgtgtaagctg

SEQ ID NO: 6 - DNA, 18 bp, Artificial Sequence
  Antisense primer for the oct4 gene
agggtttccgctttgcat

SEQ ID NO: 7 - DNA, 20 bp, Artificial Sequence
  Sense primer for the T box gene
gctgtgacaggtacccaacc

SEQ ID NO: 8 - DNA, 21 bp, Artificial Sequence
  Antisense primer for the T box gene
catgcaggtgagttgtcagaa

SEQ ID NO: 9 - DNA, 19 bp, Artificial Sequence
  Sense primer for the Mesp1 gene
ctgttggagacctggatgc

SEQ ID NO: 10 - DNA, 20 bp, Artificial Sequence
  Antisense primer for the Mesp1 gene
cgtcagttgtcccttgtcac

SEQ ID NO: 11 - DNA, 19 bp, Artificial Sequence
  Sense primer for the C/EBPa gene
gacatcagcgcctacatcg

SEQ ID NO: 12 - DNA, 18 bp, Artificial Sequence
  Antisense primer for the C/EBPa gene
ggctgtgctggaacaggt

SEQ ID NO: 13 - DNA, 20 bp, Artificial Sequence
  Sense primer for the C/EBPg gene
ggacataggagcgcaaagaa

SEQ ID NO: 14 - DNA, 20 bp, Artificial Sequence
  Antisense primer for the C/EBPg gene
ggacataggagcgcaaagaa

SEQ ID NO: 15 - DNA, 20 bp, Artificial Sequence
  Sense primer for the C/EBPb gene
ccagccccctcactaatagc

SEQ ID NO: 16 - DNA, 18 bp, Artificial Sequence
  Antisense primer for the C/EBPb gene
ccctgctctgagctgtcg

SEQ ID NO: 17 - DNA, 19 bp, Artificial Sequence
  Sense primer for the PPARg gene
cagtggggatgtctcataa

SEQ ID NO: 18 - DNA, 20 bp, Artificial Sequence
  Antisense primer for the PPARg gene
cttttggcatactctgtgat

SEQ ID NO: 19 - DNA, 20 bp, Artificial Sequence
  Sense primer for the PDGFRa gene
ccacctgagtgagattgtgg

SEQ ID NO: 20 - DNA, 21 bp, Artificial Sequence
  Antisense primer for the PDGFRa gene
tcttcaggaagtccaggtgaa

SEQ ID NO: 21 - DNA, 20 bp, Artificial Sequence
  Sense primer for the ly6E gene
gccatcctctccagaatgaa

SEQ ID NO: 22 - DNA, 19 bp, Artificial Sequence
  Antisense primer for the ly6E gene
gcaggagaagcacatcagc

SEQ ID NO: 23 - DNA, 18 bp, Artificial Sequence
  Sense primer for the CD29 gene
cgatgccatcatgcaagt

SEQ ID NO: 24 - DNA, 19 bp, Artificial Sequence
  Antisense primer for the CD29 gene
acaccagcagccgtgtaac

SEQ ID NO: 25 - DNA, 19 bp, Artificial Sequence
  Sense primer for the CD44 gene
tgccgctttgcaggtgtat

SEQ ID NO: 26 - DNA, 17 bp, Artificial Sequence
  Antisense primer for the CD44 gene
ggcctccgtccgagaga

SEQ ID NO: 27 - DNA, 18 bp, Artificial Sequence
  Sense primer for the PGC1a gene
tgagagggccaagcaaag

SEQ ID NO: 28 - DNA, 20 bp, Artificial Sequence
  Antisense primer for the PGC1a gene
ataaatcacacggcgctctt

SEQ ID NO: 29 - DNA, 18 bp, Artificial Sequence
  Sense primer for the PRDM16 gene
tggctgcttctggactca

SEQ ID NO: 30 - DNA, 26 bp, Artificial Sequence
  Antisense primer for the PRDM16 gene
atattatttacaacgtcaccgtcact

SEQ ID NO: 31 - DNA, 20 bp, Artificial Sequence
  Sense primer for the Cidea gene
tgaaggccaccatgtatgag

SEQ ID NO: 32 - DNA, 18 bp, Artificial Sequence
  Antisense primer for the Cidea gene
caggaaccgcagcagact

SEQ ID NO: 33 - DNA, 18 bp, Artificial Sequence
  Sense primer for the UCP1 gene
ctcaccgcagggaaagaa

SEQ ID NO: 34 - DNA, 20 bp, Artificial Sequence
  Antisense primer for the UCP1 gene
ggttgcccaatgaatactgc

SEQ ID NO: 35 - DNA, 20 bp, Artificial Sequence
  Sense primer for the Cited1 gene
accggacttggagtcagaga

SEQ ID NO: 36 - DNA, 20 bp, Artificial Sequence
  Antisense primer for the Cited1 gene
cagtttcgccacctgaaaac

SEQ ID NO: 37 - DNA, 19 bp, Artificial Sequence
  Sense primer for the TMEM26 gene
ttgcaccatgagacccagt

SEQ ID NO: 38 - DNA, 22 bp, Artificial Sequence
  Antisense primer for the TMEM26 gene
tgctggtattctgtgatgttcc

SEQ ID NO: 39 - DNA, 19 bp, Artificial Sequence
  Sense primer for the GAPDH gene
agccacatcgctcagacac

SEQ ID NO: 40 - DNA, 19 bp, Artificial Sequence
  Antisense primer for the GAPDH gene
gcccaatacgaccaaatcc

SEQ ID NO: 41 - DNA, 20 bp, Artificial Sequence
  Sense primer for the PPIA gene
atgctggacccaacacaaat

SEQ ID NO: 42 - DNA, 21 bp, Artificial Sequence
  Antisense primer for the PPIA gene
tctttcactttgccaaacacc

SEQ ID NO: 43 - DNA, 23 bp, Artificial Sequence
  Sense primer for the CD137 gene
agctgttacaacatagtagccac

SEQ ID NO: 44 - DNA, 22 bp, Artificial Sequence
  Antisense primer for the CD137 gene
tcctgcaatgatcttgtcctct

SEQ ID NO: 45 - DNA, 19 bp, Artificial Sequence
  Sense primer for the HOXC9 gene
gcagcaagcacaaagagga

SEQ ID NO: 46 - DNA, 21 bp, Artificial Sequence
  Antisense primer for the HOXC9 gene
cgtctggtacttggtgtaggg

SEQ ID NO: 47 - DNA, 20 bp, Artificial Sequence
  Sense primer for the PPARa gene
gcactggaactggatgacag

SEQ ID NO: 48 - DNA, 21 bp, Artificial Sequence
  Antisense primer for the PPARa gene
tttagaaggccaggacgatct

SEQ ID NO: 49 - DNA, 20 bp, Artificial Sequence
  Sense primer for the DIO2 gene
cctcctcgatgcctacaaac

SEQ ID NO: 50 - DNA, 20 bp, Artificial Sequence
  Antisense primer for the DIO2 gene
gctggcaaagtcaagaaggt

SEQ ID NO: 51 - DNA, 18 bp, Artificial Sequence
  Sense primer for the MYF5 gene
ctatagcctgccgggaca

SEQ ID NO: 52 - DNA, 23 bp, Artificial Sequence
  Antisense primer for the MYF5 gene
tggaccagacaggactgttacat

SEQ ID NO: 53 - DNA, 20 bp, Artificial Sequence
  Sense primer for the PAX7 gene
gaaaacccaggcatgttcag

SEQ ID NO: 54 - DNA, 21 bp, Artificial Sequence
  Antisense primer for the PAX7 gene
gcggctaatcgaactcactaa

SEQ ID NO: 55 - DNA, 24 bp, Artificial Sequence
  Sense primer for the CD24 gene
tgaagaacatgtgagaggtttgac

SEQ ID NO: 56 - DNA, 24 bp, Artificial Sequence
  Antisense primer for the CD24 gene
gaaaactgaatctccattccacaa

(19) United States
(12) Patent Application Publication
(10) Pub. No.: US 2017/0211043 A1
(43) Pub. Date: Jul. 27, 2017

(54) METHOD FOR IN VITRO PRODUCTION OF ADIPOCYTE PROGENITORS AND ADIPOCYTES

(71) Applicants: UNIVERSITE PIERRE ET MARIE CURIE (PARIS 6), PARIS (FR); ASSISTANCE PUBLIQUE - HÔPITAUX DE PARIS, PARIS (FR); INSERM (INSTITUT NATIONAL DE LA SANTÉ ET DE LA RECHERCHE MÉDICALE), PARIS (FR)

(72) Inventors: ANNE-CLAIRE GUENANTIN, CAMBRIDGE (GB); NOLWENN BRIAND, OSLO (NO); EMILIE CAPEL, CHEPTAINVILLE (FR); CORINNE VIGOUROUX, PARIS (FR); JACQUELINE CAPEAU, SAINT MANDE (FR)

(21) Appl. No.: 15/329,587

(22) PCT Filed: Jul. 28, 2015
(86) PCT No.: PCT/FR2015/052085

§ 371 Date: Jan. 27, 2017

(30) Foreign Application Priority Data
Jul. 29, 2014 (FR) 1457357

(51) Int. Cl. C12N 5/077; A61K 35/35

(52) U.S. Cl. CPC C12N 5/0653; A61K 35/35; C12N 2501/01; C12N 2501/155; C12N 2501/16; C12N 2501/33; C12N 2501/39; C12N 2506/45; C12N 2501/15; C12N 2501/105; C12N 2501/999

(57) ABSTRACT

The present invention relates to a method for in vitro production of adipocyte progenitors and adipocytes from pluripotent stem cells, in particular from induced pluripotent stem cells, as well as to the use of the adipocyte progenitors and adipocytes thus obtained for therapeutic or screening purposes.

The present invention relates to a novel method for in vitro production of adipocyte progenitors and adipocytes, and to therapeutic uses and screening methods using the cells thus produced.

### TECHNOLOGICAL BACKGROUND OF THE INVENTION

Stem cells are defined as cells having the ability to self-renew and to differentiate in vitro. Pluripotent stem cells, consisting of embryonic stem (ES) cells and induced pluripotent stem (iPS) cells, are able to multiply indefinitely in theory and to give rise to almost all cell types in vitro.

Induced pluripotent stem cells were developed in 2007 from human fibroblasts by the Yamanaka team (Takahashi et al., 2007). This cell type is produced by transfection of pluripotency genes (such as C-MYC, OCT3/4, SOX2 and KLF4) in somatic cells. These cells are then selected for their ability to express OCT3/4 and NANOG, two proteins involved in pluripotency (Takahashi et al., 2007; Yu et al., 2007). iPS cells have the same characteristics as ES cells in terms of their morphology, their gene expression and their epigenetic status. They are able to differentiate into the three germ layers—endoderm, ectoderm and mesoderm—in vitro and in vivo.

iPS cells have the advantage of being able to recapitulate the early stages of development, unlike the existing models derived from primary cultures of cells from patients. Moreover, they possess the entire genetic background of the donor patient, thus constituting an excellent model for studying the physiopathology of genetic diseases. Indeed, the abnormalities responsible for these pathologies can be expressed at the cellular level at various stages of differentiation of iPS cells in vitro. Lastly, iPS cells at different stages of differentiation can be used to test novel therapeutic molecules in vitro (Yamanaka, 2010), and are a hope for the development of novel cell therapies in the context of regenerative medicine.

Consequently, these cells constitute a virtually infinite source of material for studying normal or pathological cellular functions. However, the main obstacle to understanding these mechanisms is the establishment of robust and efficient protocols for producing mature cells of interest.

The adipocyte is defined as the functional unit of adipose tissue, an organ specializing in storing energy in triglyceride form. Adipose tissue constitutes the only reserve of energy that can be mobilized in the long term and thus plays a prominent role in controlling energy balance in mammals. Consequently, a defect of lipid storage within adipose tissue leads to significant metabolic disorders, the prevalence of which is constantly increasing. Moreover, the adipocyte is also an endocrine cell which produces numerous factors involved in systemic regulation, such as those of insulin sensitivity, inflammation, immune functions and blood pressure.

Although it is relatively easy to obtain human adipose tissue, the patient must undergo an invasive procedure. Moreover, mature human adipocytes taken in this way cannot be amplified and thus can be maintained in culture for only a few days.

Various groups have developed human cell models for studying adipogenesis from mesenchymal stem cells derived from bone marrow or cells derived from the stromal vascular fraction of adipose tissue (Pittenger et al. 1999; Zuk et al. 2001). However, these cell systems have certain limits, such as a reduced proliferation capacity, a decreased differentiation capacity during passaging and variable differentiation potentials. To overcome these technical problems, various methods for differentiating adipocytes from iPS cells have been developed. These protocols, however, are long and complex given that they require a preliminary step of differentiation into embryoid bodies (three-dimensional structures made up of the three germ layers) or into mesenchymal cells, which has a limited efficiency (Taura et al., 2009; Xiong et al., 2013; Noguchi et al., 2013; Ahfeldt et al., 2012). Cell differentiation efficiency can be increased by ectopic expression of adipocyte transcription factors (Ahfeldt et al. 2012), but such a protocol does not make it possible to study the physiological mechanisms brought into play during adipocyte differentiation.

It thus appears necessary to develop a simple, fast and efficient protocol for recapitulating the in vivo physiological differentiation of adipocytes, i.e., from the mesoderm, while avoiding the need to produce embryoid bodies or mesenchymal stem cells beforehand.

### SUMMARY OF THE INVENTION

The objective of the present invention is to provide to a novel method for in vitro production of adipocyte progenitors and adipocytes.

The present invention relates first to a method for in vitro production of adipocyte progenitors comprising
culturing pluripotent stem cells on an adherent culture system and in a serum-free culture medium;
contacting said pluripotent stem cells with a mesodermal differentiation medium until obtaining mesodermal progenitors; and
contacting said mesodermal progenitors with an adipogenic differentiation medium until obtaining adipocyte progenitors,

and optionally collecting the adipocyte progenitors thus obtained.

The pluripotent stem cells are preferably induced pluripotent stem cells.

The mesodermal differentiation medium is, preferably, a serum-free culture medium comprising one or more morphogens belonging to the TGF-β superfamily, in particular selected from the group consisting of activin A, activin B, BMP-4 protein, BMP-2 protein, TGF-β1, TGF-β2 and TGF-β3, and any combination thereof. According to a particular embodiment, the mesodermal differentiation medium is a serum-free culture medium comprising (i) a morphogen selected from the group consisting of activin A and activin B, preferably activin A; and (ii) a morphogen selected from the group consisting of BMP-4 protein, BMP-2 protein, TGF-β1, TGF-β2 and TGF-β3, and any combination thereof, preferably BMP-4 protein.

The serum-free culture medium is preferably a medium suitable for culturing hematopoietic cells.

The pluripotent stem cells are preferably contacted with the mesodermal differentiation medium when the culture reaches about 50% to about 90% confluence.

According to an embodiment, the adipogenic differentiation medium is a culture medium comprising insulin, one of its analogues or IGF-1, a glucocorticoid and an agent that increases intracellular cyclic adenosine monophosphate (cAMP), preferably a culture medium comprising insulin, dexamethasone and 3-isobutyl-1-methylxanthine. Preferably, the adipogenic differentiation medium further comprises indomethacin.

The present invention also relates to a method for in vitro production of adipocytes comprising contacting the adipocyte progenitors obtained by the method according to the invention with an adipocyte maturation medium until obtaining adipocytes. The adipocyte maturation medium is preferably a culture medium comprising insulin.

According to another aspect, the present invention also relates to adipocyte progenitors or adipocytes obtained by the method according to the invention, for use in the treatment of lipodystrophy, a glycemic control abnormality, preferably selected from the group consisting of fasting hyperglycemia, impaired glucose tolerance, diabetes, in particular type 2 diabetes, and insulin resistance, or dyslipidemia optionally associated with obesity or with a lipodystrophy syndrome.

The adipocyte progenitors and/or the adipocytes are preferably obtained from induced pluripotent stem cells obtained from somatic cells, preferably fibroblasts, from the subject to be treated.

The present invention also relates to a kit for in vitro production of adipocyte progenitors or adipocytes comprising a container containing one or more morphogens belonging to the TGF-β superfamily, a container containing insulin, one of its analogues or IGF-1, a glucocorticoid and an agent that increases intracellular cyclic adenosine monophosphate (cAMP) and optionally a container containing insulin.

In particular, the kit may comprise a first container containing activin A and/or BMP-4, and a second container containing insulin, dexamethasone and IBMX, and optionally indomethacin.

The present invention also relates to the use of the kit according to the invention for in vitro production of adipocyte progenitors and/or adipocytes according to the methods of the invention.

According to another aspect, the present invention also relates to a method for screening molecules that stimulate the thermogenic activity of adipocytes comprising
contacting the adipocytes obtained by the method according to the invention with candidate molecules, and
selecting the molecules that stimulate the thermogenic activity of the adipocytes.

### BRIEF DESCRIPTION OF THE DRAWINGS

FIG. 1: Schematic representation of an embodiment of the method according to the invention of adipocyte differentiation in two dimensions from iPS cells. Differentiation of the iPS cells into mesodermal precursors is induced from D0 to D4 by a differentiation medium consisting of STEMPro34 (+GlutaMAX+ascorbic acid) in the presence of activin A (25 ng/mL) and BMP4 (10 ng/mL). Differentiation into adipocytes is induced at D4 and D8 by growing the mesodermal progenitors in DMEM/F12 10% FCS, insulin (10 μg/mL), isobutylmethylxanthine (0.5 mM), dexamethasone (1 μM) and indomethacin (50 μM). The adipocytes are then maintained in DMEM/F12 10% FCS 1 μg/mL insulin to allow their maturation.

FIG. 2: Pluripotent nature of iPS cells. (A) Phase contrast image of a pluripotent iPS colony (10×). (B) Photographs of iPS cell colonies after alkaline phosphatase staining (wide field, left panel; 10× magnification, right panel). (C) Immunofluorescence labeling of the pluripotency markers NANOG, SOX2, OCT4, SSEA3/4, TRA-1-60 and TRA-1-81. (D) Comparison of gene expression of the pluripotency markers OCT4, NANOG and SOX2 between iPS cells and H9 embryonic stem cells (n=3).

FIG. 3: Differentiation of iPS cells into mesodermal and adipocyte progenitors. Relative expression level (arbitrary units) measured by real-time quantitative PCR of the specific pluripotency markers NANOG, SOX2 and OCT4 (n=3) (A), of the mesoderm, namely BRACHYURY (T BOX) and MESP1 (B), at 0, 2, 4 and 6 days of differentiation (n=3). Immunofluorescence labeling of adipocyte progenitors using the antibodies MESP1 and BRACHYURY (C). Relative expression level (arbitrary units) measured by real-time quantitative PCR of BRACHYURY (T box) and MESP1 (D) during differentiation of iPS cells into mesenchymal stem cells (n=3). Relative expression level (arbitrary units) measured by real-time quantitative PCR of PDGFRα, LY6E, CD44, CD29 and CD24. *p<0.05, p determined by the Mann-Whitney nonparametric test with the GraphPad Prism software (E) (n=3). Immunofluorescence labeling of adipocyte progenitors using the CD44, PDGFRα and CD29 and Ki67 antibodies (F).

FIG. 4: Differentiation of mesodermal progenitors into adipocytes. Relative expression level (arbitrary units) measured by real-time quantitative PCR of the four adipocyte transcription factors (A) C/EBPβ (n=3), (B) C/EBPδ (n=3), (C) C/EBPα (n=3) and (D) PPARγ (n=3), during adipocyte differentiation.

FIG. 5: Characterization of adipocytes after 20 days of differentiation. (A) Photographs of adipocytes after Oil Red O staining (wide field, left; 20×, middle; 40×, right). (B) Immunofluorescence labeling of the markers C/EBPα (top) and GLUT4 (bottom) in adipocytes at 20 days of differentiation. Lipid droplets are stained with Nile Red and nuclei with DRAQ5. (C) Protein expression of PPARγ1 and PPARγ2, C/EBPα p30/42, insulin receptor β-subunit (IR-β), perilipin 1 and caveolin 1 in iPS cells at D0, D10 and D20. β-Actin is used as the control (n≧3). (D) Phosphorylation of the insulin receptor β-subunit (IR-β) and AKT/PKB was evaluated following a short treatment with insulin in iPS cells after 20 days of differentiation using phospho-specific and total antibodies.

FIG. 6: Production of beige adipocytes. Relative gene expression of the markers of classic brown adipocytes (A) PGC1α (n=3), PRDM16 (n=3) and UCP1 (n=3). (B) Detection of UCP1 expression by Western blot at days 0, 10 and 20 of differentiation. β-Actin is shown as a deposition control. (C) Relative gene expression of the beige adipocyte specific markers TMEM26, CITED1, CD137 and HOXC9 during differentiation. (n≧3). *p<0.05 vs D4. (D) Immunofluorescence labeling of the beige adipocyte marker CITED1 in adipocytes at 20 days of differentiation. Lipid droplets are stained with Nile Red and nuclei with DRAQ5. (E) Detection of UCP1 expression by Western blot under basal conditions or after 6 hours of 10⁻⁵ M isoproterenol. β-Actin is shown as a deposition control. (F) Staining of beige adipocytes with MitoTracker® and with BODIPY under basal conditions (top) and after 48 hours of 8-Br-cAMP (bottom). (G) Gene expression of the genes involved in thermogenesis PGC1α, PPARα, PRDM16 and DIO2 of iPS cells after 20 days of differentiation under basal conditions (untreated) and after 48 hours of 8-Br-cAMP *p<0.05 vs Basal.

FIG. 7: Formation of adipose tissue in vivo following transplant of adipocytes derived from iPS cells in nude mice. (A) Schematic representation of the transplant of adipocytes derived from iPS cells in nude mice. (B) Macroscopic view of the panniculus adiposus formed from adipocytes derived from iPS cells. (C) Hematoxylin and eosin staining of the panniculus adiposus formed from adipocytes derived from iPS cells (left) and from MSC (right). Scale bar: 500 μm. (D) High magnification of hematoxylin and eosin staining of the panniculus adiposus formed from adipocytes derived from iPS cells (left) and from MSC (right). Scale bar 200 μm (top) and 100 μm (bottom). The arrows indicate blood vessels. (E) Labeling with anti-perilipin 1 antibody of the panniculus adiposus formed from adipocytes derived from iPS cells (left) and from MSC (right) at various magnifications. Scale bar 200 μm (top) and 100 μm (bottom).

### DETAILED DESCRIPTION OF THE INVENTION

The inventors have developed a simple, fast and efficient method for producing large quantities of adipocytes from pluripotent stem cells, preferably induced pluripotent stem cells, in only 20 days. Indeed, they have shown that in the presence of a medium comprising mesodermal differentiation inducers, stem cells were able to differentiate directly—i.e., without first forming embryoid bodies or mesenchymal stem cells—into mesodermal progenitors able, in turn, to produce adipocyte progenitors and adipocytes in the presence of an adipogenic cocktail.

The inventors observed that the adipocytes obtained by the method according to the invention expressed specific adipocyte markers and had a morphology characteristic of this cell type. They also showed that the cells obtained had the characteristics of beige-type adipocytes, for which no human model has been described to date.

Thus, according to a first aspect, the present invention relates to a method for in vitro production of adipocyte progenitors comprising
culturing pluripotent stem cells in an adherent culture system and in a serum- free culture medium;
contacting said pluripotent stem cells with a mesodermal differentiation medium until obtaining mesodermal progenitors; and
contacting said mesodermal progenitors with an adipogenic differentiation medium until obtaining adipocyte progenitors.

As used in this document, the term “pluripotent stem cells” includes embryonic stem cells and reprogrammed somatic cells (or induced pluripotent stem cells). Preferably, this term refers to cells from a mammal, in particular from a mouse, a rat or a primate, and more particularly from a human.

Embryonic stem cells are derived from the inner cell mass of blastocysts and have the ability to give rise to all tissues of the organism (mesoderm, endoderm, ectoderm), including germline cells. The pluripotency of embryonic stem cells can be evaluated by the presence of markers such as the transcription factors OCT3/4, NANOG and SOX2 and surface markers such as SSEA3/4, Tra-1-60 and Tra-1-81. This pluripotency can also be confirmed in vivo by the formation of teratomas in mice (Rossant et al., 1982). Embryonic stem cells can be obtained without destruction of the embryo from which they are derived, for example using the technique described by Chung et al. (2008). In a particular embodiment, and for legal or ethical reasons, the embryonic stem cells are nonhuman embryonic stem cells.

As used in this document, the term “reprogrammed somatic cells”, “induced pluripotent stem cells”, “iPSCs” or “iPS” refers to pluripotent cells obtained by genetic reprogramming of differentiated somatic cells. In addition to their morphology and their self-renewal and pluripotency potential being similar to those of embryonic stem cells, iPSCs also exhibit epigenetic reprogramming with an overall histone methylation and gene expression profile very close to that of embryonic stem cells. iPSCs are in particular positive for pluripotency markers, in particular alkaline phosphatase staining and expression of NANOG, SOX2, OCT4 and SSEA3/4 proteins.

The methods for producing iPSCs are well-known to the person skilled in the art and are in particular described in the articles by Yu et al. (2007), Takahashi et al. (2007) and Nakagawa et al. (2008). In particular, iPSCs can be obtained from human somatic cells transfected with the transcription factors Oct3/4, Sox2, Klf4 and c-Myc (Takahashi et al., 2007), Oct3/4, Sox2, Nanog and Lin28 (Yu et al., 2007) or with the Oct3/4, Sox2 and Klf4 genes (Nakagawa et al., 2008). iPSCs can be obtained from a wide variety of cells such as fibroblasts, B-lymphocytes, keratinocytes or meningeal membrane cells (Patel et al., 2010). Preferably, the iPSCs used in the method according to the invention are obtained from fibroblasts, in particular human fibroblasts. According to a particular embodiment, the iPSCs are obtained from fibroblasts from a lipodystrophic patient.

The method according to the invention comprises a step of culturing pluripotent stem cells, as defined above, in an adherent culture system and in a serum-free culture medium. These culture conditions differ from those used for the formation of embryoid bodies which requires the use of non-adherent culture systems in order to allow the stem cells to aggregate.

The adherent culture system suitable to be used in the method according to the invention may be an adherent monolayer culture system or a culture system on feeder cells.

The culture system may be in any form suited to the method according to the invention, in particular in the form of a flask, a multiwell plate or a dish.

According to an embodiment, the adherent culture system is a culture system on feeder cells which promote the proliferation and/or control the differentiation of cells with which they are co-cultured. Preferably, these feeder cells stimulate the proliferation of cells in culture without inducing their differentiation. They are frequently irradiated to prevent their proliferation and the invasion of the culture of interest. The feeder cells suitable to be used in the method according to the invention may be easily selected by the person skilled in the art from the various known types, such as mouse embryonic fibroblast (MEF) cells or human foreskin cells (see the patent application EP 2182052).

According to a preferred embodiment, the adherent culture system is an adherent monolayer culture system. This system comprises a solid support, for example glass or plastic, usually coated with a matrix or a substrate promoting cell adherence.

The substrate may be a protein substrate consisting of attachment factors and promoting the adhesion of cells to the support. These attachment factors may in particular be selected from poly-L-lysine, collagen, fibronectin, laminin or gelatin.

The matrices that mimic the extracellular matrix and are suitable to be used in the method according to the invention are well-known to the person skilled in the art and many varieties are available commercially. These matrices comprise, for example, matrices of the Matrigel™, Geltrex® type or other matrices comprising one or more anchoring proteins such as collagen, laminin, fibronectin, elastin, proteoglycans, aminoglycans or vitronectin. Three-dimensional hydrogel-type matrices may also be used. According to a preferred embodiment, the matrix is of the Matrigel™ type.

The pluripotent stem cells are cultured in a serum-free medium making it possible to propagate and to maintain the cells in an undifferentiated state. Numerous media of this type are available commercially and are well-known to the person skilled in the art (see for example the article by Chen et al., 2011). The serum-free culture medium may be, for example, mTESR1™ medium (STEMCELL Technologies), E8 medium (Life Technologies) or hPSC medium (Promocell). According to a preferred embodiment, the culture medium used does not comprise serum of animal origin.

The cells are preferably subcultured regularly to prevent the culture from reaching confluence, i.e., from covering the entire available surface. Indeed, confluence induces a cessation of proliferation and unwanted metabolic changes. The cells may be subcultured using standard techniques well-known to the person skilled in the art. They may in particular be detached from the matrix or the support by the action of enzymes such as collagenase IV or by mechanical passage in PBS or any other enzyme-free solution containing EDTA (e.g.: ReleSR, (STEMCELL Technologies)), collected by centrifugation, dissociated mechanically and reseeded in a new culture system.

In the method according to the invention, the pluripotent stem cells cultured on the adherent culture system and in the serum-free medium are contacted with a mesodermal differentiation medium until obtaining mesodermal progenitors.

Optionally, cell confluence may be measured or evaluated before the stem cells are contacted with the mesodermal differentiation medium. Preferably, the stem cells are contacted with the mesodermal differentiation medium when the cell culture reaches about 50% to about 90% confluence. The person skilled in the art is familiar with the concept of cell confluence and is capable of evaluating it by any known method. By way of example, the term “90% confluence” may be defined as the situation wherein colonies come into contact with other colonies, while a space representing about 10% of the entire surface area remains unoccupied between the colonies. As used herein, the term “about” refers to a range of values of ±10% of the specified value. For example, “about 20” includes 20±10%, or 18 to 22.

According to a particular embodiment, the stem cells are contacted with the mesodermal differentiation medium for one to three days, preferably for two days, after being placed in culture on the adherent culture system and in the serum-free medium.

Preferably, the contacting is carried out by simply changing the culture medium. Alternatively, it may be carried out by subculturing in an adherent culture system as described above comprising the mesodermal differentiation medium.

According to an embodiment, the adherent culture system is a culture system on feeder cells as described above. The feeder cells able to be used may be easily selected by the person skilled in the art from the various known types, such as mouse embryonic fibroblast (MEF cells) or human foreskin cells (see the patent application EP 2182052), preferably in the presence of an FGF signaling pathway inhibitor, such as the compound SU5402 (Mohammadi et al. 1997).

The cells may be subcultured using standard techniques well-known to the person skilled in the art as described above, in particular may be detached from the matrix or the support by the action of enzymes such as collagenase IV, by mechanical passage in PBS or any other enzyme-free solution containing EDTA (e.g.: ReleSR (STEMCELL Technologies) or by the action of a commercial cell detachment medium such as TrypLE™ Express (Life Technologies), collected by centrifugation, dissociated mechanically and reseeded in a new culture system.

Alternatively, the stem cells may be contacted with the mesodermal differentiation medium once placed in culture on the adherent culture system for a period of about 4 days.

The mesodermal differentiation medium is a culture medium which allows both the survival and the proliferation of cells but also which induces or promotes the differentiation of cells into mesodermal progenitors. Preferably, this medium prevents or limits the differentiation of cells into other cell types, in particular into endodermal or ectodermal progenitors.

According to a particular embodiment, the mesodermal differentiation medium is a serum-free basal culture medium comprising one or more morphogens belonging to the TGF-β superfamily.

Preferably, the serum-free basal culture medium is a culture medium suited to the proliferation of human hematopoietic cells (CD34+). This medium may be a minimum medium particularly comprising mineral salts, amino acids, vitamins and a carbon source essential to cells; a buffer system for regulating pH. Preferably, this medium further comprises bovine serum albumin; transferrin or iron; selenium; insulin or an analogue thereof; and/or a glucocorticoid such as hydrocortisone or dexamethasone.

The media able to be used in the method according to the invention comprise for example, but are not limited to, StemPro-34® medium (Invitrogen) or any other medium described in the patent application WO 97/033978, TeSR™-E6 medium (STEMCELL™ Technologies), the media described in the patent U.S. Pat. No. 5,945,337, or MethoCult™ medium (STEMCELL™ Technologies).

Depending on the medium used, it may be necessary or desirable to add glutamine (this amino acid is unstable and usually must be added extemporaneously), vitamin C (which oxidizes quickly) and/or one or more antibiotics.

The morphogen(s) belonging to the TGF-β superfamily are preferably selected from the group consisting of activin A, activin B, BMP-4 protein, BMP-2 protein, TGF-β1, TGF-β2 and TGF-β3, and any combination thereof.

According to an embodiment, the mesodermal differentiation medium comprises (i) a morphogen selected from the group consisting of activin A and activin B; and (ii) a morphogen selected from the group consisting of BMP-4 protein, BMP-2 protein, TGF-β1, TGF-β2 and TGF-β3, and any combination thereof.

According to another embodiment, the mesodermal differentiation medium comprises activin A and BMP-4 protein.

Preferably, the medium comprises between 1 and 25 ng/mL BMP-4, more preferably about 10 ng/mL BMP-4.

Preferably, the medium comprises between 5 and 100 ng/mL activin A, more preferably about 25 ng/mL activin A.

According to a particularly preferred embodiment, the mesodermal differentiation medium comprises about 10 ng/mL BMP-4 and about 25 ng/mL activin A.

According to a particular embodiment, the mesodermal differentiation medium comprises the complete serum-free medium StemPro-34® (Invitrogen) enriched in glutamine, or an equivalent culture medium, BMP-4, preferably about 10 ng/mL, activin A, preferably about 25 ng/mL, and optionally ascorbic acid.

The pluripotent stem cells are maintained in the differentiation medium until obtaining mesodermal progenitors. During this period, and in a conventional manner, the culture medium may be changed regularly, preferably every day or every two days.

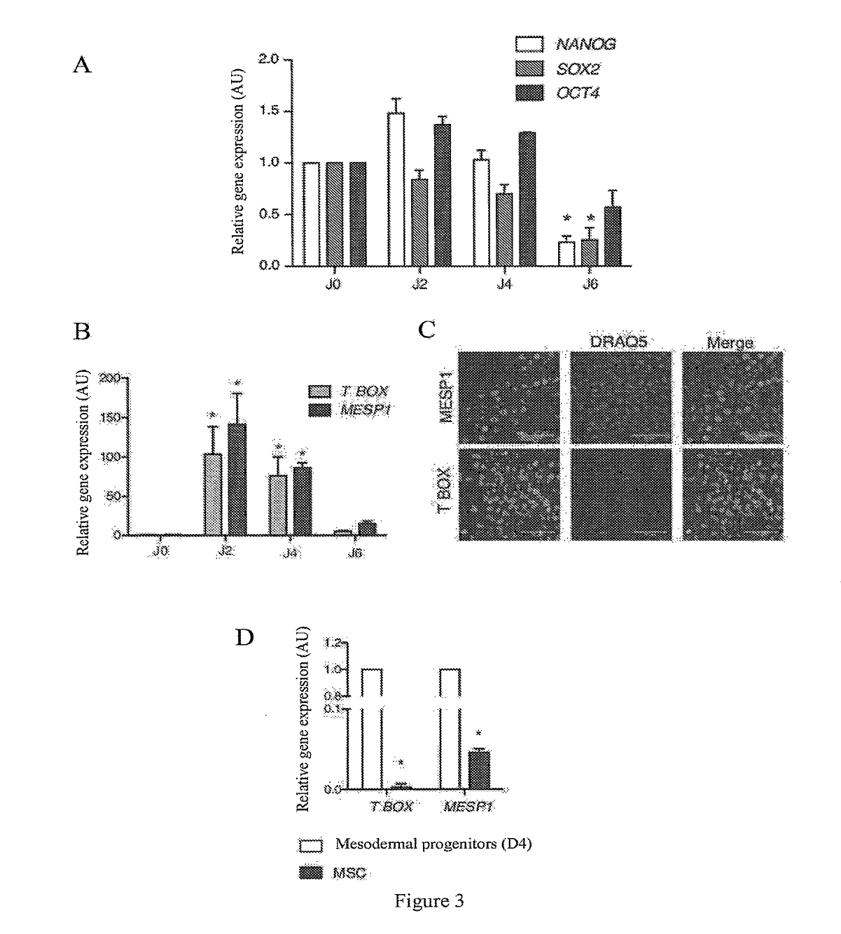

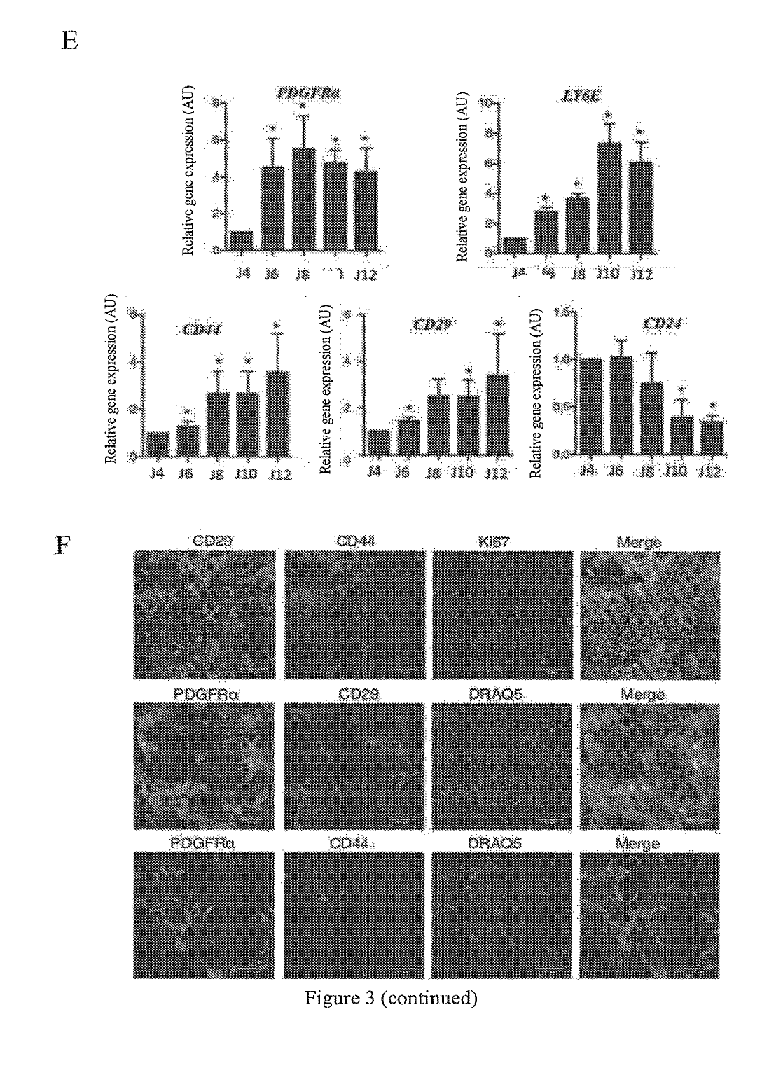

As used in this document, the term “mesodermal progenitors” or “mesodermal precursors” refers to cells, preferably human cells, able to differentiate (without preliminary dedifferentiation or reprogramming) into most mesodermal tissues, in particular into endothelial cells, adipocytes, cardiomyocytes, osteogenic cells, chondrocytes, mesenchymal cells and hematopoietic cells. These cells are characterized by the expression of the BRACHYURY (T BOX) (Gene ID: 6862) and MESP1 (Gene ID: 55897) genes, two genes specific to early mesoderm. This characteristic differentiates them from mesenchymal stem cells or cells derived from the stromal vascular fraction of adipose tissue which do not express the BRACHYURY and MESP1 genes (FIG. 3D).

The appearance of mesodermal progenitors can be easily detected by the person skilled in the art by monitoring expression of the BRACHYURY and MESP1 genes. Indeed, as illustrated in the experimental section and in FIG. 3B, these genes are not expressed in pluripotent stem cells. This expression is also correlated with a decrease in expression of the pluripotency markers NANOG and SOX2 (FIG. 3A).

The protein expression of BRACHYURY and MESP1 can be measured easily by the person skilled in the art by an immunofluorescence method as illustrated in the experimental section and in FIG. 3C.

Thus, optionally, the method according to the invention may comprise an additional step consisting of measuring or evaluating the expression of the BRACHYURY gene and/or the MESP1 gene. The expression of these markers may be monitored by any technique known to the person skilled in the art, for example by real-time quantitative PCR.

According to a particular embodiment, the pluripotent stem cells are contacted with the mesodermal differentiation medium for 2 to 5 days, preferably for 3 to 4 days, and more preferably for 4 days.

The mesodermal progenitors thus obtained are then contacted with an adipogenic differentiation medium until obtaining adipocyte progenitors.

As before, this contacting may be carried out by simply changing the culture medium or by subculturing in an adherent culture system comprising the adipogenic differentiation medium.

According to an embodiment, the adherent culture system is a culture system on feeder cells as described above. The feeder cells able to be used may be easily selected by the person skilled in the art from the various known types, such as mouse embryonic fibroblast (MEF cells) or human foreskin cells (see the patent application EP 2182052), preferably in the presence of an FGF signaling pathway inhibitor, such as the compound SU5402 (Mohammadi et al. 1997).

The cells may be subcultured using standard techniques well-known to the person skilled in the art as described above, in particular may be detached from the matrix or the support by the action of enzymes such as collagenase IV, by mechanical passage in PBS or any other enzyme-free solution containing EDTA (e.g.: ReleSR (STEMCELL Technologies) or by the action of a commercial cell detachment medium such as TrypLE™ Express (Life Technologies), collected by centrifugation, dissociated mechanically and reseeded in a new culture system. Preferably, the mesodermal progenitors are contacted with an adipogenic differentiation medium by simply changing the culture medium.

The adipogenic differentiation medium, also called adipocyte differentiation medium, is a culture medium which allows both the survival and the proliferation of mesodermal progenitors but which also induces or promotes the differentiation of these cells into adipocyte progenitors.

According to an embodiment, the adipogenic differentiation medium is a culture medium comprising insulin, an insulin analogue or IGF-1, a glucocorticoid and an agent that increases intracellular cyclic adenosine monophosphate (cAMP). Preferably, the adipogenic differentiation medium comprises insulin or an analogue thereof, a glucocorticoid and an agent that increases intracellular cyclic adenosine monophosphate (cAMP).

The insulin analogues may be selected for example from the group consisting of NPH insulin (Eli Lilly), lispro (Eli Lilly), aspart (Novo Nordisk) and glulisine (Sanofi-Aventis).

The glucocorticoid may be selected for example from the group consisting of dexamethasone, betamethasone, cortivazol and hydrocortisone. Preferably, the glucocorticoid is dexamethasone.

The agent that increases intracellular cyclic adenosine monophosphate (cAMP) may be any compound known to increase the intracellular concentration of cAMP. This agent may in particular be selected from the group consisting of phosphodiesterase inhibitors, direct activators of protein kinase A (or cAMP-dependent protein kinase) and adenylate cyclase activators.

Phosphodiesterase inhibitors comprise, but are not limited to, methylated xanthines and derivatives thereof such as 3-isobutyl-1-methylxanthine (IBMX), caffeine, aminophylline, paraxanthine, pentoxifylline, theobromine and theophylline.

Direct activators of protein kinase A comprise, but are not limited to, belinostat (PXD101), adrenalin, glucagon, and cAMP analogues such as 8-bromo-cAMP.

Adenylate cyclase activators comprise, but are not limited to, forskolin, glucagon, prostaglandins D2, E1 and I2, carbacyclin, dopamine, endothelin 1, L-epinephrine and parathyroid hormone.

According to a preferred embodiment, the adipogenic differentiation medium is a culture medium comprising insulin, dexamethasone and 3-isobutyl-1-methylxanthine.

Preferably, the medium comprises between 1 and 20 μg/mL insulin, more preferably about 10 μg/mL insulin.

Preferably, the medium comprises between 0.0001 and 500 mM IBMX, more preferably between 0.01 and 10 mM IBMX, and still more preferably between 0.1 and 1 mM IBMX. According to a particular embodiment, the medium comprises between about 0.1 mM and about 0.5 mM IBMX, preferably about 0.5 mM IBMX.

Preferably the medium comprises between 0.25 and 100 μM dexamethasone, more preferably about 1 μM dexamethasone.

According to a particular embodiment, the adipogenic differentiation medium is a culture medium comprising about 10 μg/mL insulin, about 1 μM dexamethasone and about 0.5 mM IBMX.

Optionally, the differentiation medium may also comprise one or more additional compounds promoting adipocyte differentiation such as indomethacin, a compound of the thiazolidinedione family such as pioglitazone or rosiglitazone, the growth factor FGF21, irisin, triiodothyronine, retinoic acid, BMP7 and/or BMP8, in particular indomethacin, a compound of the thiazolidinedione family such as pioglitazone or rosiglitazone, growth factor FGF21, irisin, triiodothyronine and/or retinoic acid. Preferably, the differentiation medium further comprises indomethacin, preferably 0.01 to 0.5 mM indomethacin, and more preferably about 0.1 mM indomethacin. According to a particular embodiment, the differentiation medium further comprises 50 μM indomethacin. Thus, according to a preferred embodiment, the adipogenic differentiation medium is a culture medium comprising insulin, preferably about 10 μg/mL, dexamethasone, preferably about 1 μM, IBMX, preferably about 0.5 mM, and indomethacin, preferably about 0.1 mM. According to another preferred embodiment, the adipogenic differentiation medium is a culture medium comprising insulin, preferably about 10 μg/mL, dexamethasone, preferably about 1 μM, IBMX, preferably about 0.5 mM, and indomethacin, preferably about 0.05 mM.

The basal culture medium used in the adipocyte differentiation medium is, preferably, a basal synthetic minimum medium particularly including mineral salts, amino acids, vitamins and a carbon source essential to cells, and a buffer system for regulating pH. The media able to be used in the method according to the invention include, for example, but are not limited to, DMEM/F12 medium, DMEM medium, RPMI medium, Ham's F12 medium, IMDM medium and KnockOut™ DMEM medium (Life Technologies).

The medium is preferably supplemented with 2 to 20%, preferably 5 to 15%, serum, in particular fetal calf serum.

The mesodermal progenitors are maintained in the adipocyte differentiation medium until obtaining adipocyte progenitors. During this period, and in a conventional manner, the culture medium may be changed regularly, preferably every two or three days.

As used in this document, the term “adipocyte progenitors”, “pre-adipocytes” or “adipocyte stem cells” refers to proliferative cells, i.e., expressing a cell proliferation marker, preferably Ki67, which express adipose tissue stem cell markers including CD44 (Gene ID: 960), CD29 (Gene ID: 3688), PDGFRα (Gene ID: 5156) and LY6E (Gene ID: 4061) (Zuk, 2013). Preferably, these cells are negative for the antigens CD31 (Gene ID: 5175) and CD34 (Gene ID: 947). In the presence of an adipogenic cocktail, these cells are able to differentiate (without preliminary dedifferentiation or reprogramming) into adipocytes.

The appearance of adipocyte progenitors may be easily detected by the person skilled in the art by monitoring the expression of adipose tissue stem cell markers such as CD44, CD29, PDGFRα and LY6E. Indeed, as illustrated in the experimental section and in FIGS. 3E and F, these markers are only very weakly expressed in mesodermal progenitors. The difference in expression is in particular very large for the marker PDGFRα which has an expression level in adipocyte progenitors about 8 times higher than in mesodermal progenitors. The expression of these markers may be monitored by any technique known to the person skilled in the art, for example by real-time quantitative PCR.

Thus, optionally, the method according to the invention may comprise an additional step consisting of measuring or evaluating the expression of one or more of the markers CD44, CD29, PDGFRα and LY6E.

According to a particular embodiment, the mesodermal progenitors are contacted with the adipocyte differentiation medium for 2 to 5 days, preferably for 3 to 4 days, and more preferably for 4 days.

The method according to the invention may comprise a step of collecting the adipocyte progenitors obtained. This collecting may be carried out using standard techniques well-known to the person skilled in the art. The progenitors may in particular be detached from the matrix or the support by the action of enzymes such as collagenase IV or a commercial cell detachment solution such as TryPLE™ Express (Life Technologies). They may then be isolated on the basis of various markers such as for example CD44 or CD29.

The adipocyte progenitors may then be reseeded on matrices that mimic the extracellular matrix as described above, well-known to the person skilled in the art. Many varieties are available commercially. These matrices may comprise feeder cells such as mouse embryonic fibroblast (MEF cells) or human foreskin cells (see the patent application EP 2182052) preferably in the presence of an FGF signaling pathway inhibitor, such as the compound SU5402 (Mohammadi et al. 1997). The culture system used is preferably an adherent monolayer culture system. This system comprises a solid support, for example glass or plastic, usually coated with a matrix or a substrate promoting cell adherence. The substrate may be a protein substrate consisting of attachment factors and promoting the adhesion of cells to the support. These attachment factors may in particular be selected from poly-L-lysine, collagen, fibronectin, laminin or gelatin.

Alternatively, the adipocyte progenitors obtained by the method according to the invention may be contacted with an adipocyte maturation medium until obtaining adipocytes.

The present invention thus also relates to a method for in vitro production of adipocytes comprising contacting the adipocyte progenitors obtained by the method according to the invention with an adipocyte maturation medium until obtaining adipocytes.

As before, this contacting may be carried out by simply changing the culture medium or by subculturing in an adherent culture system as described above comprising the adipocyte maturation medium. Preferably the adipocyte progenitors are contacted with an adipocyte maturation medium by simply changing the culture medium.

The adipocyte maturation medium is a culture medium which allows both the survival and the proliferation of adipocyte progenitors but also which induces or promotes the differentiation of these cells into adipocytes.

The basal culture medium used in the adipocyte maturation medium may be the same basal culture medium as that used in the adipocyte differentiation medium. Alternatively, it may be different.

According to an embodiment, the basal culture medium used in the adipocyte maturation medium is a basal synthetic minimum medium particularly including mineral salts, amino acids, vitamins and a carbon source essential to cells, and a buffer system for regulating pH. The media able to be used in the method according to the invention comprise for example, but are not limited to, DMEM/F12 medium, DMEM medium, RPMI medium, Ham's F12 medium, IMDM medium, and KnockOut™ DMEM medium (Life Technologies).

This medium is preferably supplemented with 2 to 20%, preferably 5 to 15%, serum, in particular fetal calf serum.

According to a preferred embodiment, the adipocyte maturation medium comprises, or consists essentially of, a basal culture medium supplemented with insulin, and optionally with serum. Preferably, the medium comprises 0.1 to 5 μg/mL insulin, more preferably about 1 μg/mL insulin.

The adipocyte progenitors are maintained in the adipocyte maturation medium until obtaining adipocytes. During this period, and in a conventional manner, the culture medium may be changed regularly, preferably every two or three days.

As used in this document, the term “adipocytes” refers to cells characterized by the gene expression of C/EBPβ (Gene ID: 1051), C/EBPδ (Gene ID: 1052), C/EBPα (Gene ID: 1050) and PPARγ (Gene ID: 5468) and by an accumulation of neutral lipids in the form of lipid droplets detectable by Oil Red staining. The adipocytes may also be characterized by the expression of the insulin receptor (Gene ID: 3667), perilipin 1 (Gene ID 5346), caveolin 1 (Gene ID: 857) or the glucose transporter GLUT4 (Gene ID: 442992).

Furthermore, the inventors observed that the adipocytes obtained by the method according to the invention also expressed markers of brown adipocytes such as the PGC1 (Gene ID: 10891), PRDM16 (Gene ID: 63976) and UCP1 (Gene ID: 7350) genes but also specific markers of beige adipocytes such as TMEM26 (Gene ID: 219623), CITED1 (Gene ID: 4435), CD137 (Gene ID: 3604) and HOXC9 (Gene ID: 3225).

The appearance of adipocytes may be easily detected by the person skilled in the art by monitoring the expression of markers specific to adipocytes, to brown adipocytes and to beige adipocytes as defined above, preferably by monitoring the markers C/EBPδ, PPARγ, CITED1 and PGC1α. The expression of these markers may be monitored by any technique known to the person skilled in the art, for example by real-time quantitative PCR. Alternatively, the appearance of adipocytes may be easily detected by staining the cells with Oil Red.

Thus, optionally, the method according to the invention may comprise an additional step consisting of measuring or evaluating the expression of one or more of the markers C/EBPδ, PPARγ, CITED1 and PGC1α and/or monitoring the appearance of adipocytes by staining the cells with Oil Red.

According to a particular embodiment, the adipocyte progenitors are contacted with the adipocyte maturation medium for 5 to 20 days or for 5 to 15 days, preferably for 10 to 12 days, and more preferably for 12 days.

The present invention also relates to the adipocyte progenitors and the adipocytes obtained by the method according to the invention.

It also relates to a pharmaceutical composition comprising the adipocyte progenitors and/or the adipocytes obtained by the method according to the invention, and one or more pharmaceutically acceptable excipients.

The pharmaceutically acceptable excipients must be compatible with the cells and may be, for example, a culture medium, a buffer solution or a saline solution. In particular, the composition may comprise Matrigel™ or an equivalent excipient.

In a preferred embodiment, the pharmaceutical composition is suitable for parenteral administration, preferably by subcutaneous route, in particular for administration directly in adipose tissue. The pharmaceutical composition may be formulated in accordance with the standard pharmaceutical practices known to the person skilled in the art.

In a particular embodiment, the pharmaceutical composition comprises the adipocyte progenitors and/or the adipocytes obtained by the method according to the invention, encapsulated in a biocompatible matrix. Many encapsulation technologies may be used, particularly those described in the document WO 91/10425.

The pharmaceutical composition may also comprise one or more additional active compounds, for example compounds known to improve cell survival or proliferation or to prevent contamination.

According to still another aspect, the present invention relates to the therapeutic use of the adipocyte progenitors and/or the adipocytes obtained by the method according to the invention, in particular for the treatment of lipodystrophies or metabolic disorders.

The present invention thus relates to the adipocyte progenitors and/or the adipocytes obtained by the method according to the invention, for use in the treatment of lipodystrophy or metabolic disorders. It also relates to a pharmaceutical composition according to the invention for use in the treatment of lipodystrophy or metabolic disorders.

The present invention also relates to the use of the adipocyte progenitors and/or the adipocytes obtained by the method according to the invention, for the preparation of a drug intended for the treatment or the prevention of lipodystrophy or metabolic disorders.

The present invention further relates to a method for treating lipodystrophy or a metabolic disorder comprising administering to the subject to be treated a therapeutically effective amount of the adipocyte progenitors and/or the adipocytes obtained by the method according to the invention. Preferably, the subject to be treated is human.

As used herein, the term “metabolic disorder” refers to glycemic control abnormalities, in particular fasting hyperglycemia, impaired glucose tolerance, diabetes, in particular type 2 diabetes, or insulin resistance, or to dyslipidemia optionally associated with obesity or lipodystrophy syndrome. A patient may be regarded as obese when his BMI is greater than 25, preferably greater than 28, and more preferably greater than 30.

Lipodystrophies are disorders characterized by a selective loss of adipose tissue from various regions of the body. The extent of the fat loss may range from very small regions to the near-total absence of adipose tissue in the entire body. The problems encountered by patients may be purely esthetic or may lead to severe metabolic complications, overall proportional to the degree of adipose loss.

Lipodystrophies are classified according to the general or partial nature of the fat loss, and whether known genetic factors are involved or not. Lipodystrophies of genetic origin are congenital or delayed-onset monogenic disorders. Several genes responsible for hereditary lipodystrophies have been identified, such as, for example, genes coding for A-type lamins, AGPAT2, caveolin-1, cavin-1, seipin, PPARg, perilipin, CIDEC or Akt2 (Guenantin et al., 2014). Acquired lipodystrophies may be the consequence of drug treatments (particularly antiviral therapies or insulin injections or other drugs) or mostly dysimmune diseases (for example Lawrence and Barraquer-Simons syndromes).

The principal lipodystrophies leading to metabolic disorders (representing lipodystrophic syndromes) are generalized lipodystrophy of genetic origin (CGL for congenital generalized lipodystrophy) or Berardinelli-Seip syndrome; partial lipodystrophy of genetic origin (FPLD for familial partial lipodystrophy); Lawrence-type acquired generalized lipodystrophy syndrome, Barraquer-Simons partial lipodystrophy syndrome, lipodystrophy linked to HIV infection and to antiretroviral therapies; multisystemic syndromes comprising lipodystrophy such as CANDLE-type auto-inflammatory syndromes (JASP, JMP, or Nakajo syndrome) linked to PSMB8 gene mutations, Hutchinson-Gilford progeria and other progeroid syndromes including mandibuloacral dysplasia, linked to mutations of lamins A/C or ZMPSTE24, Werner-type progeria linked to mutations of WRN protein, syndromic dwarfism with lipoatrophy associated with PCYT1A (phosphate cytidylyltransferase 1 alpha) mutations, microcephalic dwarfism associated with NSMCE2 mutations, and other syndromes comprising lipodystrophy of yet unknown cause.

According to an embodiment, somatic cells, preferably fibroblasts, from the patient to be treated are reprogrammed in order to produce iPSCs. Adipocyte progenitors and/or adipocytes are then obtained by the method according to the invention, from these iPSCs, before being administered to the patient, preferably by subcutaneous injection. According to a particular embodiment, the treatment method according to the invention thus comprises producing adipocyte progenitors and/or adipocytes from induced pluripotent cells obtained from somatic cells from the patient to be treated, and administering the adipocyte progenitors and/or the adipocytes thus obtained to the patient.

In the case of lipodystrophies caused by a genetic mutation, particularly for congenital lipodystrophies, the mutation at the origin of the lipodystrophy may be detected and corrected according to methods well-known to the person skilled in the art, for example by homologous recombination or by genetic engineering methods based on ZFN, TALEN or CRISPR/Cas (Gaj et al., 2013). This correction is preferably carried out before proliferation and differentiation of the iPSCs. The adipocyte progenitors and/or the adipocytes obtained by the method according to the invention from these “corrected” iPSCs are then administered to the patient.

The adipocyte progenitors and/or the adipocytes obtained by the method according to the invention may be used in the treatment of lipodystrophies to fill in regions of the body depleted by the loss of adipose material. In this case, the adipocyte progenitors and/or the adipocytes are preferably injected subcutaneously directly in the region to be filled in.

When the adipocyte progenitors and/or the adipocytes obtained by the method according to the invention are used in the treatment of metabolic disorders, they are preferably injected subcutaneously, in particular directly in the adipose tissue, in order to increase the proportion of adipocytes having thermogenic activity or able to have this activity, for example after induction by a thermogenic stimulus.

The present invention also relates to a method for treating lipodystrophy or a metabolic disorder, comprising administering to a subject, preferably human, a therapeutically effective amount of the adipocyte progenitors and/or the adipocytes obtained by the method according to the invention.

The term “treatment” as used in this document refers to an improvement or disappearance of symptoms, a slowing of the progression of the disease, a cessation of the evolution of the disease or a disappearance of the disease. This term also includes both preventive and curative treatment.

The term “therapeutically effective amount” as used herein refers to a sufficient amount to have an effect on at least one esthetic symptom (filling) or metabolic symptom of the lipodystrophy or the metabolic disorder (restoring the metabolic activity of adipose tissue).

The present invention also relates to a kit for in vitro production of adipocyte progenitors or adipocytes.

This kit comprises:
a first container containing one or more compounds present in the mesodermal differentiation medium as described above, preferably one or more morphogens belonging to the TGF-β superfamily, in particular one or more morphogens selected from the group consisting of activin A, activin B, BMP-4 protein, BMP-2 protein, TGF-β1, TGF-β2, TGF-β3 and any combination thereof,
a second container containing one or more compounds present in the adipogenic differentiation medium as described above, preferably insulin, one of its analogues or IGF-1, a glucocorticoid and an agent that increases intracellular cyclic adenosine monophosphate (cAMP), and more preferably insulin, dexamethasone, 3-isobutyl-1-methylxanthine, and optionally indomethacin; and
optionally a third container containing one or more compounds present in the adipogenic maturation medium as described above, preferably insulin.

Preferably, the kit comprises containers each comprising one or more compounds at a concentration or in an amount that facilitates the reconstitution and/or the use of the differentiation and/or maturation medium and the implementation of the method according to the invention.

The kit according to the invention may also comprise a container containing a basal medium used in the mesodermal differentiation medium as described above, a container containing a basal medium used in the adipogenic differentiation medium as described above or a container containing the basal culture medium used in the adipocyte maturation medium as described above.

In a particular embodiment, the kit comprises a container containing a mesodermal differentiation medium as described above, a container containing an adipogenic differentiation medium as described above and optionally a container containing an adipocyte maturation medium as described above.

According to another particular embodiment, the kit comprises
a first container containing activin A and/or BMP-4 protein, and optionally a serum-free basal culture medium, preferably suited to the proliferation of human hematopoietic cells;
a second container containing insulin, dexamethasone and IBMX, and optionally a basal culture medium, preferably a basal synthetic minimum medium optionally containing serum; and
optionally a third container containing insulin and optionally a basal culture medium, preferably a basal synthetic minimum medium optionally containing serum.

The second container may further comprise indomethacin.

The kit according to the invention may also comprise an adherent culture system, in particular in the form of a flask, a multiwell plate or a dish.

The kit may also comprise instructions indicating the methods for preparing and/or using the differentiation or maturation media for in vitro production of adipocyte progenitors or adipocytes according to the method of the invention.

The present invention also relates to the use of the kit according to the invention for in vitro production of adipocyte progenitors and/or adipocytes according to the methods of the invention.

According to another aspect, the present invention relates to the use of the adipocyte progenitors and/or the adipocytes obtained by the method according to the invention for screening molecules of therapeutic interest.

The molecules of therapeutic interest may be in particular molecules activating the beige phenotype of adipocytes, and more particularly molecules increasing the thermogenic activity of adipose tissues. These molecules may be particularly usable in the treatment or the prevention of metabolic disorders as described above.

The present invention thus relates to a method for screening molecules of interest comprising
contacting the adipocyte progenitors and/or the adipocytes obtained by the method according to the invention with candidate molecules, and
selecting the molecules having the desired activity.

The present invention relates in particular to a method for screening molecules that stimulate the thermogenic activity of adipocytes comprising
contacting the adipocytes obtained by the method according to the invention with one or more candidate molecules, and
selecting the molecules that stimulate the thermogenic activity of the adipocytes.

The thermogenic activity of the adipocytes may be evaluated by techniques well-known to the person skilled in the art, such as, for example, indirect evaluation methods comprising measuring the oxygen consumption of cells (Oxoplate® or Seahorse technologies).

Depending on the nature of the molecules sought, the pluripotent stem cells used to produce the adipocyte progenitors and/or the adipocytes may be obtained from a healthy subject or a subject having a defined pathology, for example a subject having a metabolic disorder as defined above.

All the references cited in this description are incorporated by reference in the present application. Other features and advantages of the invention will become more apparent upon reading the following examples given by way of non-limiting illustration.

### EXAMPLES

### Materials and Methods

Culture of Human Induced Pluripotent Stem (iPS) Cells

The technique used to reprogram human fibroblasts into iPS cells is that one from the protocol published by Yamanaka et al. (Takahashi et al., 2007), modified using a viral vector (Sendai virus). iPS cells from control subjects were cultured on Matrigel™ (hESC Matrigel, BD Biosciences, cat. no. 3542777) and the propagation medium mTESR1™ (STEMCELL™ Technologies, cat. no. 05850) was changed daily. The cells were subjected every 4 days to collagenase type IV (Gibco) (45 minutes at 37° C.) at a concentration of 1 mg/mL then centrifuged at 800 rpm for 4 minutes. The cells were then resuspended in mTESR1™ and the clones were isolated mechanically using a 5 mL pipette. Clusters of about 20 cells were then seeded on a dish incubated beforehand with Matrigel™.

### Adipocyte Differentiation

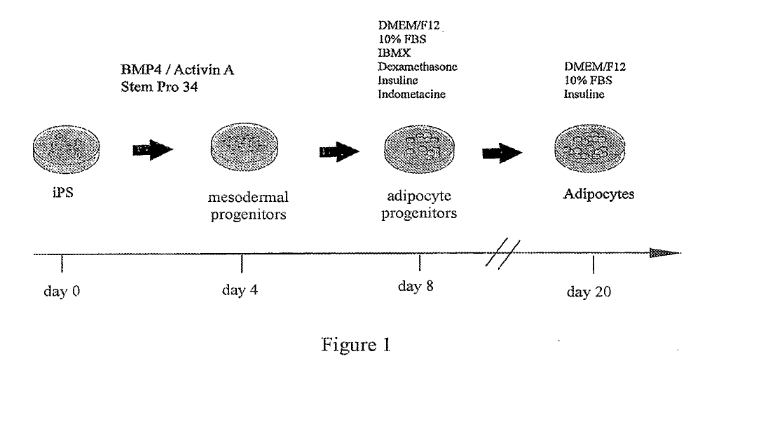

A schematic representation of an embodiment according to the invention is presented in FIG. 1.

After enzymatic separation and mechanical dissociation, the iPS cells were seeded on Matrigel™. After one or two days of culture in mTESR1™, reaching 70% confluence, the cells were placed, at the time defined as D0, in differentiation medium for producing mesodermal progenitors: complete STEMPro34 (Life Technologies, cat. no. 10639011) enriched with 2 mM GlutaMAX (Invitrogen, cat. no. 35050061), 10 μg/mL ascorbic acid (Sigma, cat. no. A4403), 10 ng/mL BMP4 (R&D Systems, cat. no. 314-LP) and 25 ng/mL activin A (R&D Systems, cat. no. 338-AC). This mesodermal induction medium was changed at D2.

At D4, adipocyte differentiation of the mesodermal progenitors was induced using the differentiation medium DMEM/F12 10% FCS supplemented with 10 μg/mL insulin (Sigma, cat. no. I9278), 0.5 mM isobutylmethylxanthine (Sigma, cat. no. 15879), 1 μM dexamethasone (Sigma, cat. no. D4902) and 50 μM indomethacin (Sigma, cat. no. 17378). At D7, the culture medium was changed with the same medium. Then, to allow maturation of the adipocytes, the cells were cultured in DMEM/F12 10% FCS supplemented with 1 μg/mL insulin until D20.

### Immunofluorescence

The cells were fixed with 3% PFA for 15 minutes at room temperature. The nonspecific sites were blocked by incubating the cells in 3% PBS-BSA. The cell preparation was incubated in the presence of a monoclonal or polyclonal antibody specific to the protein of interest (Table 1 below), diluted in a solution of PBS, 0.1% Triton X100 or 0.1% saponin, 3% BSA. The protein-antibody complex thus formed was detected by incubating the cells for 1 hour at room temperature away from light with a secondary antibody which is covalently coupled to a fluorochrome and diluted in a solution of PBS, 0.01% Triton X100 or saponin, 3% BSA. The anti-rabbit IgG or anti-mouse IgG secondary antibodies were coupled to Alexa 488 (Invitrogen, 1:1000). The preparation was then incubated for 15 minutes with 25 ng/mL Nile Red (Molecular Probes®, N-1142) then rinsed 3 times with PBS before being incubated for 5 minutes in DAPI (4′-6-diamidino-2-phenylindole, VWR). After applying the mounting medium (Fluoromount-G™, Southern Biotech), the cells were observed using a confocal microscope (Leica).

TABLE 1
Antibodies used

| Antibodies | Item number |
|---|---|
| OCT4 | Bovision 3576 |
| SOX2 | Milipore AB5603 |
| NANOG | Cell signaling D73G4 |
| SSEA3/4 | R&D MAB1435 |
| TRA-1-60 | MAB4360-Millipore |
| TRA-1-81 | MAB4381-Millipore |
| MESP1 | Abcam Ab77013 |
| T BOX | R&D AF2085 |
| CD29 | BD Biosciences PE 555443 |
| CD44 | BD Biosciences PE 550989 |
| PDGFRA | Cell signaling D1E1E |
| KI67 | AbCam Ab15580 |
| C/EBPA | Santa Cruz 14A4 |
| GLUT4 | Santa Cruz H61 |
| PPARg | Santa Cruz 7273 |
| IRB | Santa Cruz 29B4 |
| PERILIPIN1 | Progen GP29 |
| CAVEOLIN1 | BD Biosciences 610059 |
| P-PY | Santa Cruz PY99 |
| P-AKT | Santa Cruz Ser473 |
| AKT | Santa Cruz H136 |
| UCP1 | Abcam 23841 |
| CITED1 | Cell signaling 5H6 |
| BACTIN | Sigma A5441 |

### Alkaline Phosphatase Staining

The cells were fixed with 95% ethanol for 15 minutes at room temperature. After rinsing 3 times with PBS, the cells were then incubated for 5 minutes at 37%, 5% CO₂ with SigmaFAST™ BCPI®/NBT solution (cat. no. B5655).

### Oil Red Staining of Neutral Lipids

Oil Red staining was carried out on the adipocytes after 20 days of differentiation. After rinsing with 1×PBS, the cells were fixed in 4% (w/v) paraformaldehyde for 1 hour then incubated for 2 hours in a solution of “Oil Red O” (Sigma) diluted in isopropanol. The cells were then rinsed 4 times with tap water.

### Real-Time Quantitative PCR

Total RNAs were extracted using the NucleoSpin RNA kit (Macherey Nagel) according to the manufacturer's recommendations. The concentration of total RNAs extracted and their contamination by solvents or salts were evaluated by microspectrophotometry (Nanodrop). Reverse transcription was carried out using the High Capacity cDNA Reverse Transcription Kit (Applied Biosystems). Quantitative PCR was then carried out by adding 2 μL of cDNA diluted 10 times, 10 μL of SYBR Green I PCR mix (Roche Diagnostics, including DNA polymerase, dNTPs, 3 mM MgCl₂ and SYBR Green fluorescent probe), 0.2 μM sense primer and 0.2 μM antisense primer (Table 2 below). The reference gene GAPDH was used to normalize the expression of the genes of interest.

TABLE 2
Primers used

| Gene | Sense primer | Antisense |
|---|---|---|
| NANOG | atgcctcacacggagactgt | cagggctgtcctgaataagc |
| | (SEQ ID NO: 1) | (SEQ ID NO: 2) |
| SOX2 | gggggaatggaccttgtatag | gcaaagctcctaccgtacca |
| | (SEQ ID NO: 3) | (SEQ ID NO: 4) |
| OCT3/4 | gcttcaagaacatgtgtaagctg | agggtttccgctttgcat |
| | (SEQ ID NO: 5) | (SEQ ID NO: 6) |
| T BOX | gctgtgacaggtacccaacc | catgcaggtgagttgtcagaa |
| | (SEQ ID NO: 7) | (SEQ ID NO: 8) |
| MESP1 | ctgttggagacctggatgc | cgtcagttgtcccttgtcac |
| | (SEQ ID NO: 9) | (SEQ ID NO: 10) |
| C/EBPα | gacatcagcgcctacatcg | ggctgtgctggaacaggt |
| | (SEQ ID NO: 11) | (SEQ ID NO: 12) |
| C/EBPδ | ggacataggagcgcaaagaa | ggacataggagcgcaaagaa |
| | (SEQ ID NO: 13) | (SEQ ID NO: 14) |
| C/EBPβ | ccagccccctcactaatagc | ccctgctctgagctgtcg |
| | (SEQ ID NO: 15) | (SEQ ID NO: 16) |
| PPARγ | cagtggggatgtctcataa | cttttggcatactctgtgat |
| | (SEQ ID NO: 17) | (SEQ ID NO: 18) |
| PDGFRα | ccacctgagtgagattgtgg | tcttcaggaagtccaggtgaa |
| | (SEQ ID NO: 19) | (SEQ ID NO: 20) |
| LY6E | gccatcctctccagaatgaa | gcaggagaagcacatcagc |
| | (SEQ ID NO: 21) | (SEQ ID NO: 22) |
| CD29 | cgatgccatcatgcaagt | acaccagcagccgtgtaac |
| | (SEQ ID NO: 23) | (SEQ ID NO: 24) |
| CD44 | tgccgctttgcaggtgtat | ggcctccgtccgagaga |
| | (SEQ ID NO: 25) | (SEQ ID NO: 26) |
| PGC1α | tgagagggccaagcaaag | ataaatcacacggcgctctt |
| | (SEQ ID NO: 27) | (SEQ ID NO: 28) |
| PRDM16 | tggctgcttctggactca | atattatttacaacgtcaccgtcact |
| | (SEQ ID NO: 29) | (SEQ ID NO: 30) |
| CIDEA | tgaaggccaccatgtatgag | caggaaccgcagcagact |
| | (SEQ ID NO: 31) | (SEQ ID NO: 32) |
| UCP1 | ctcaccgcagggaaagaa | ggttgcccaatgaatactgc |
| | (SEQ ID NO: 33) | (SEQ ID NO: 34) |
| CITED1 | accggacttggagtcagaga | cagtttcgccacctgaaaac |
| | (SEQ ID NO: 35) | (SEQ ID NO: 36) |
| TMEM26 | ttgcaccatgagacccagt | tgctggtattctgtgatgttcc |
| | (SEQ ID NO: 37) | (SEQ ID NO: 38) |
| GAPDH | agccacatcgctcagacac | gcccaatacgaccaaatcc |
| | (SEQ ID NO: 39) | (SEQ ID NO: 40) |
| PPIA | atgctggacccaacacaaat | tcificactttgccaaacacc |
| | (SEQ ID NO: 41) | (SEQ ID NO: 42) |
| CD137 | agctgttacaacatagtagccac | tcctgcaatgatcttgtcctct |
| | (SEQ ID NO: 43) | (SEQ ID NO: 44) |
| HOXC9 | gcagcaagcacaaagagga | cgtctggtacttggtgtaggg |
| | (SEQ ID NO: 45) | (SEQ ID NO: 46) |
| PPARα | gcactggaactggatgacag | tttagaaggccaggacgatct |
| | (SEQ ID NO: 47) | (SEQ ID NO: 48) |
| DIO2 | cctcctcgatgcctacaaac | gctggcaaagtcaagaaggt |
| | (SEQ ID NO: 49) | (SEQ ID NO: 50) |
| MYF5 | ctatagcctgccgggaca | tggaccagacaggactgttacat |
| | (SEQ ID NO: 51) | (SEQ ID NO: 52) |
| PAX7 | gaaaacccaggcatgttcag | gcggctaatcgaactcactaa |
| | (SEQ ID NO: 53) | (SEQ ID NO: 54) |
| CD24 | tgaagaacatgtgagaggtttgac | gaaaactgaatctccattccacaa |
| | (SEQ ID NO: 55) | (SEQ ID NO: 56) |

Differentiation of iPSCs Into Mesenchymal Stem Cells (MSCs)

The iPSCs were mechanically passaged then seeded on gelatin (Sigma) in a differentiation medium consisting of KnockOut DMEM (Invitrogen), 20% FCS, 1% nonessential amino acids, 1% GlutaMAX (Invitrogen), 50 μM β-mercaptoethanol (Sigma), 10 ng/mL FGF2 (Peprotech), 1 mM AA2P (Sigma) (P0). After 10 to 15 days of culture, the cells were passaged with trypsin (Gibco) and diluted to 1/2 (P1). When 90% confluence was reached, the cells were passaged and diluted to 1/3 (P2). For the following passages, the cells were seeded on gelatin at 8000 cells per cm². After 6 to 7 passages, a homogeneous and stable population of MSCs was obtained.

### β-Adrenergic Stimulation of Adipocytes

The adipocytes were optionally treated for 6 hours with 10⁻⁵ M isoproterenol (Sigma), then the protein samples were collected. For a longer stimulation, the adipocytes were treated with a non-metabolizable cAMP analogue, 8-Br-cAMP (Sigma), for 48 hours.

### MitoTracker Staining of Adipocytes

Living cells at D20 of differentiation were incubated with 1 μM MitoTracker® Red CMXRos (Life Technologies®) for 45 minutes in the dark at 37° C., 5% CO₂. After rinsing twice with PBS, the cells were fixed with 3% PFA for 15 minutes at room temperature. The preparation was then incubated for 15 minutes with 1 ng/mL BODIPY 493/503 (Molecular Probes®, D-3922) then rinsed 3 times with PBS before being incubated for 5 minutes in DAPI (4′-6-diamidino-2-phenylindole, VWR). After applying the mounting medium (Fluoromount-G™, Southern Biotech), the cells were observed using a confocal microscope (Leica).

### Western Blot

The adipocytes were lysed in a suitable volume of lysis buffer (50 mM Tris pH 7.4, 0.27 M sucrose, 1 mM Na-orthovanadate pH 10, 1 mM EDTA, 1 mM EGTA, 10 mM (3-glycerophosphate, 50 mM NaF, 5 mM pyrophosphate, 1% (w/v) Triton X-100, 0.1% (w/v) 2-β-mercaptoethanol, and protease inhibitors). The total lysates were centrifuged (15,000 g, 4° C. for 10 minutes) then stored at −80° C. until use. The protein concentrations were determined using the Bradford method with bovine albumin as the standard. The samples underwent SDS/PAGE migration on polyacrylamide gels and then were transferred onto nitrocellulose membranes (Amersham Biosciences), blocked for 2 hours at room temperature in TBS-T buffer (50 mM Tris-HCl pH 7.6, 150 mM NaCl, 0.1% (v/v) Tween-20) supplemented with 5% (w/v) skimmed milk or BSA, and incubated with the various antibodies specific to the protein of interest (Table 1 above).

The nitrocellulose membranes were rinsed 3×5 minutes in TBS-T buffer before being incubated with the secondary antibody coupled to peroxidase. The signals were detected using chemiluminescence (Pierce-Perbio Biotechnologies) by exposure on autoradiographic film (Kodak).

### Standard Karyotyping

Karyotypes were produced using the standard methods for karyotyping G-bands and R-bands.

### Adipocyte Transplant In Vivo

At 18 days of culture, the cells were collected using TrypLE Express (Life Technologies, no. 12604021) and resuspended in DMEM/F12/Matrigel medium comprising 10 μg/mL insulin, 500 μM IBMX, 1 μM dexamethasone and 50 μM indomethacin. 10⁷ cells were injected subcutaneously into the back of 6-week-old Foxn1^{nu} nude mice (Taconic). These mice were also injected in the sternum with 3×10⁷ mesenchymal stem cells (MSCs) derived from iPS cells or with Matrigel alone as control. The mice are euthanized 30 days after the transplant.

### Hematoxylin and Eosin (HE) Staining

The newly formed human panniculus adiposa are excised, fixed in 4% PFA, embedded in paraffin, then cut into 4 μm sections. After deparaffinization, the slides are stained in automated systems. The slides are incubated for 5 minutes in hematoxylin, rinsed with running water, incubated for 2 minutes in eosin solution, rinsed with running water, plunged in two successive absolute alcohol baths then in toluene before being mounted with resin.

Immunolabeling with Anti-Perilipin1 Antibody: Immunohistochemistry

After deparaffinization, the antigenic sites are unmasked as a function of the primary antibody by heating (15 minutes at 95° C.) in a water bath in EDTA buffer (pH 8 or pH 9) or in a microwave in citrate buffer (pH 6). Incubation for 5 minutes in the presence of hydrogen peroxide (3%) allows the inhibition of endogenous peroxidases. The blocking of nonspecific sites is carried out by incubating the slides for 20 minutes in Dako universal serum.

The primary antibody (anti-perilipinl, Progen, Mab to Perilipin/PLIN1, cat. no. 651156), 1:500 dilution in Bond Primary Antibody Diluent (Dako), is incubated for 1 hour at room temperature. After rinsing several times successively with Dako wash buffer, the slides are incubated for 30 minutes with an HRP secondary antibody (anti-guinea pig, 1:100 in Antibody Diluent). The immunohistochemical labeling is developed by 3 incubations of 5 minutes with AEC reagent after rinsing the slides (3-amino-9-ethylcarbazole kit, Vector Laboratories). The development is stopped by plunging the slides into running water. The slides are then counterstained with Hemalum and coverslips are mounted in aqueous mounting medium (Glycergel Mounting Medium, Dako).

### Results

### Pluripotent Nature of the iPS Cell Line Used

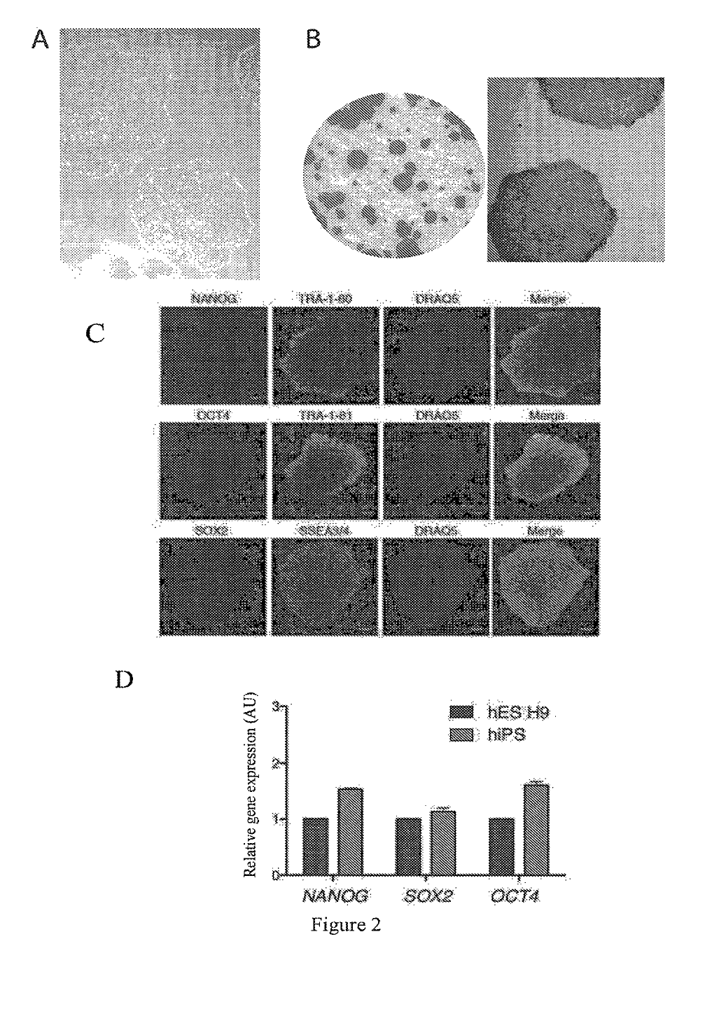

FIG. 2 shows that the iPS cells used have the expected pluripotency characteristics. The cells are organized in tight colonies with well-defined borders (FIG. 2A, 2B). They are positive for pluripotency markers such as alkaline phosphatase staining (FIG. 2B) and express NANOG, SOX2, OCT4, TRA-1-60, TRA-1-81 and SSEA3/4 proteins (FIG. 2C). Moreover, these cells express OCT4, NANOG and SOX2 genes at a level comparable to that of embryonic stem cells of the H9 line, showing their pluripotent nature (FIG. 2D).

### Production of Mesodermal and Adipocyte Progenitors

FIG. 3 shows the drop in the expression of the pluripotency markers OCT4, NANOG and SOX2 during differentiation (FIG. 3A) and induction of gene expression of BRACHYURY and MESP 1, two genes characterizing the early mesoderm, when the iPS cells are subjected to specific mesodermal differentiation medium (FIG. 3B). Gene expression of BRACHYURY (T BOX) and MESP1 is increased by a factor of 75 after 4 days of differentiation, showing the efficient production of mesodermal progenitors. The protein expression of these markers was confirmed by immunofluorescence (FIG. 3C). As expected, the expression of these genes decreases after induction of adipocyte differentiation (D6) (FIG. 3B). In order to show the specific nature of BRACHYURY and MESP1 expression in the mesodermal progenitors, the expression of these same markers was analyzed during the differentiation of iPS cells into mesenchymal stem cells (MSCs) (FIG. 3D). These markers are not expressed in MSCs and the mesodermal progenitors obtained by the method according to the invention are different from MSCs.

These mesodermal progenitors then differentiate into adipocyte progenitors that express certain markers described for stem cells from human adipose tissue. Indeed, between 4 and 12 days of differentiation, PDGFRα, LY6E, CD44 and CD29 gene expression increases (FIG. 3E). In parallel, CD24 expression decreases during differentiation in accordance with engagement toward the adipocyte pathway (FIG. 3E). The markers CD44, CD29, and PDGFRα are co-expressed at the protein level at D12 (FIG. 3F). Moreover, these same cells are positive for the marker Ki67 (FIG. 3F), indicating the presence of a proliferative progenitor population.

### Adipocyte Differentiation

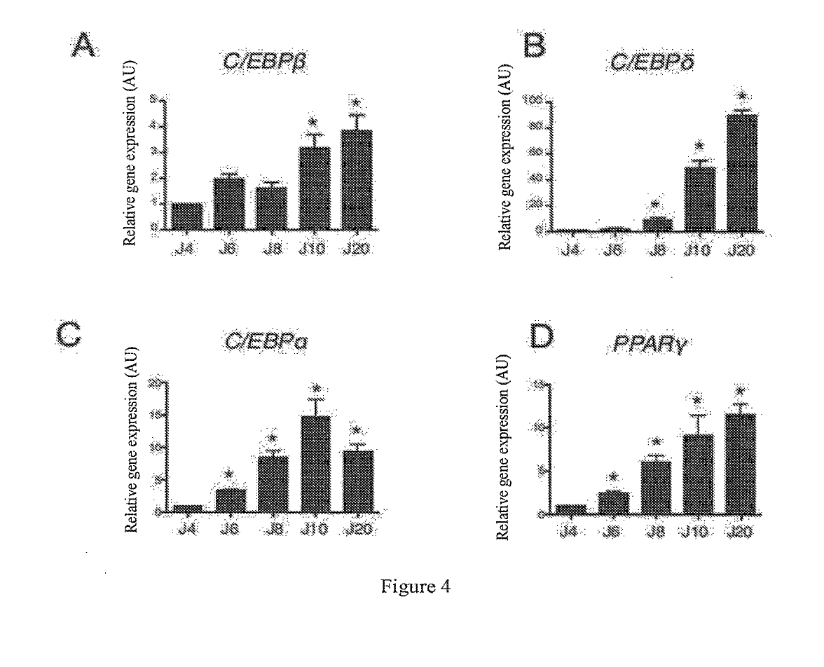

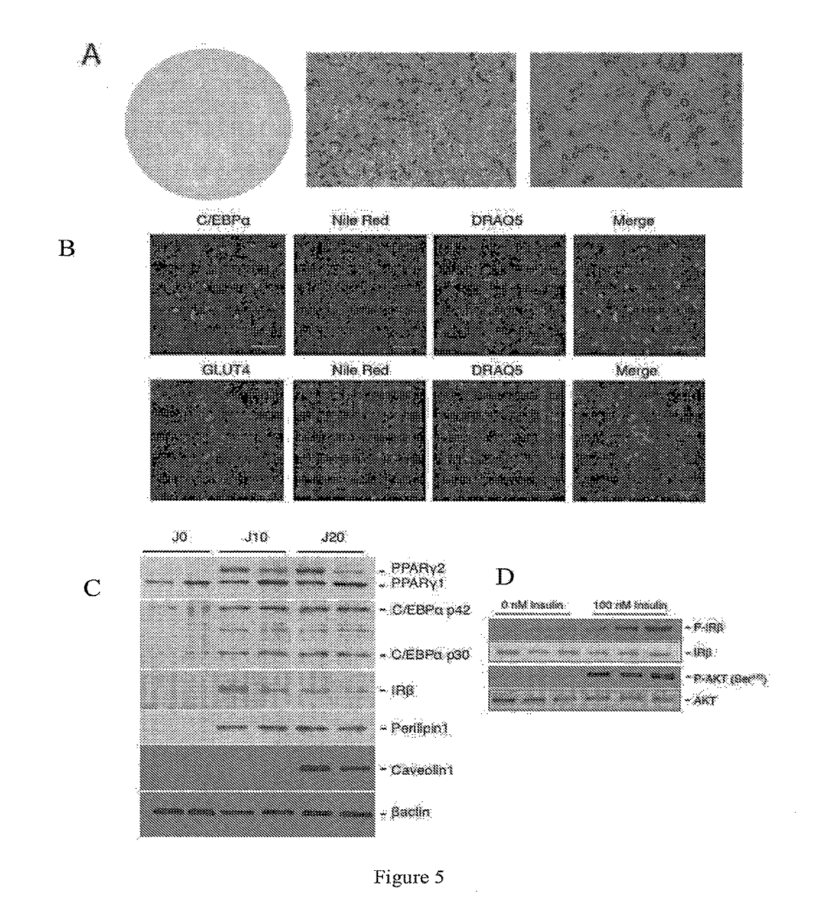

The expression of specific adipocyte genes is measured during differentiation. FIG. 4 shows that the gene expression of C/EBPα, C/EBPβ, C/EBPβ and PPARγ, transcription factors induced during adipocyte differentiation gradually increases between D8 and D12, after addition of the adipogenic differentiation cocktail (D4). This high expression level is maintained at D20. Likewise, protein expression of the adipose-specific isoform PPARγ2 and of C/EBPα p30/42 is observed from the tenth day of differentiation (FIG. 5B and 5C). FIG. 5C shows that the expression of proteins playing an important role in the adipocyte—such as the insulin receptor (IR), the proteins associated with lipid droplets such as Perilipinl and Caveolinl (FIG. 5C), and the glucose transporter GLUT4 (FIG. 5B)—are induced during the differentiation of adipocyte cells.

### Production of Adipocytes

FIG. 5A shows Oil Red staining of the adipocytes at various magnifications. Lipid accumulation is homogeneous over the entire culture dish. The cells are organized in clusters, display round shapes and contain several lipid droplets. These cells thus have a morphology characteristic of the adipocyte in vitro.

FIG. 5B presents another method of labeling neutral lipids, with concomitant labeling of the nucleus. The images show the off-center nucleus of the adipocyte, and the characteristic organization of the lipid droplets.

### Differentiation Efficiency

FIG. 5A shows extensive fields of adipocytes whose lipid droplets have been labeled with a neutral lipids stain. These images make it possible to evaluate differentiation efficiency as being greater than 60%.

Indeed, after 20 days of differentiation, 62% (±2% SEM) of the cells express in their nucleus the adipocyte marker C/EBPα. The adipocytes obtained are thus able to respond to a short insulin treatment by inducing strong phosphorylation of the insulin receptor (IR) α-subunit and its target AKT/PK (FIG. 5D).

### Production of Beige Adipocytes

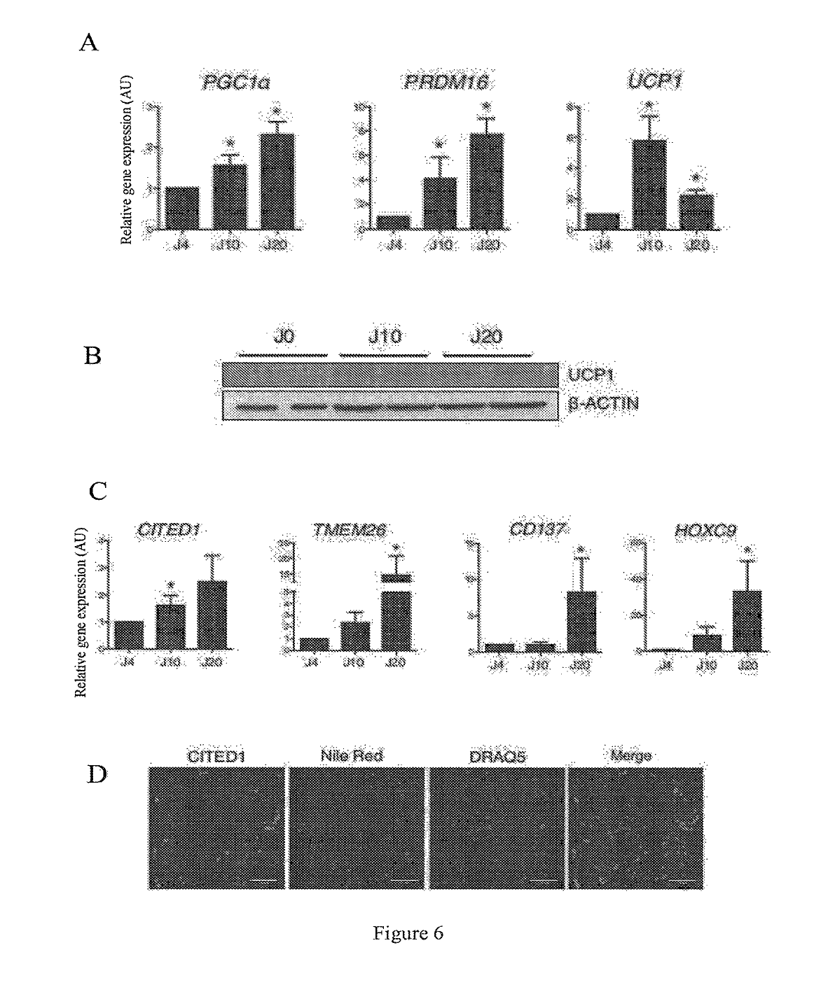

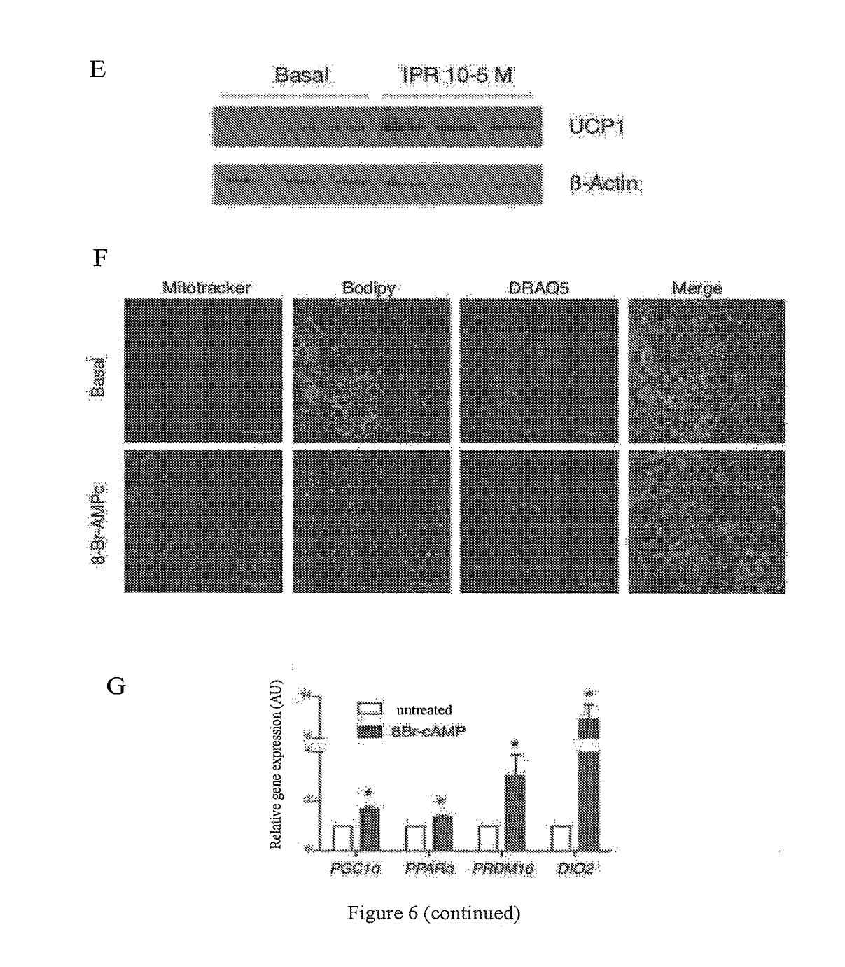

FIG. 6A shows that the adipocytes obtained express “classic brown” genes such as PGC1α, PRDM16 and UCP1, but do not express genes specific to progenitors and to mature brown adipocytes such as MYF5 and ZIC1. UCP1 protein expression can be detected as of D10 (FIG. 6B). These cells also express genes specific to beige (or “brite”) adipocytes such as TMEM26, CITED1, CD137 and HOXC9 (FIG. 6C). CITED1 protein expression can be observed in the differentiated adipocytes (FIG. 6D). Moreover, strong UCP1 protein induction is observed after β-adrenergic stimulation. This strong induction is one of the principal characteristics of beige adipocytes (FIG. 6E). Consistent with this result, stimulation with 8-Br-cAMP shows an increase in the number of mitochondria in the beige adipocytes after 48 hours (FIG. 6F) and in the expression of genes involved in thermogenesis such as PGC1α, PRDM16, PPARα and DIO2 (FIG. 6G). Thus, all these results show that the adipocytes derived from human iPS cells have a beige-type phenotype.

### Adipose Tissue Formation In Vivo

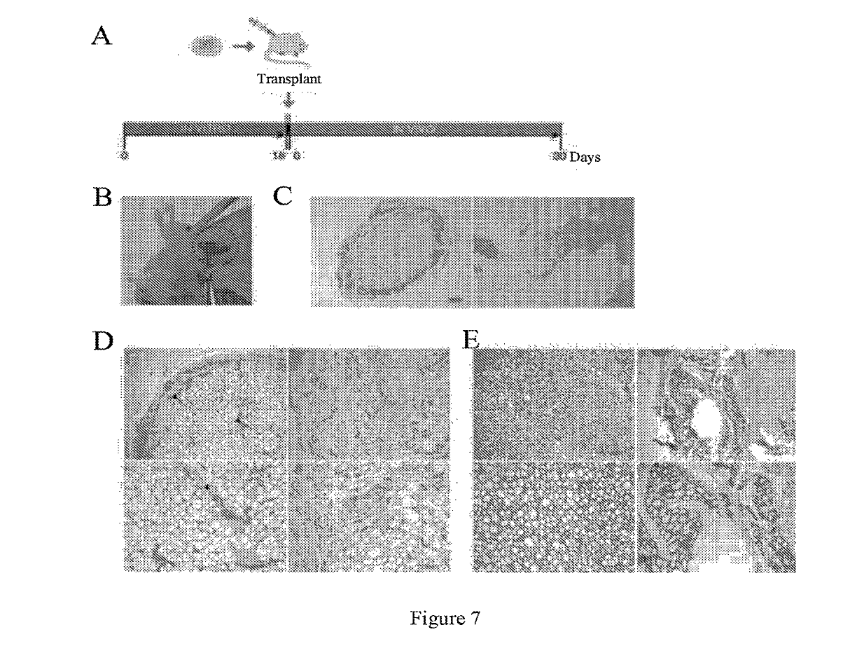

After 18 days of culture on Matrigel™ in the presence of an adipogenic differentiation medium, 10⁷ cells are injected subcutaneously into the back of six-week-old immunodeficient mice (FIG. 7A). MSCs or Matrigel™ alone are/is injected in the sternum of the same mice as the control (n=3). After 30 days, a panniculus adiposus appears at the cell injection site and not at the Matrigel™ injection site. Histological analysis of the panniculus adiposa formed after the injection of adipocytes derived from human iPS cells reveals adipose tissue which is fully differentiated, organized and vascularized (FIG. 7C, D). On the other hand, the tissues formed following injection with mesenchymal stem cells (MSCs) derived from iPS cells have a heterogeneous composition with extensive regions of fibroblastic-type cells and a smaller number of adipocytes (FIG. 7C, D). The cells that make up the panniculus adiposus formed from the adipocytes or the MSCs derived from iPS cells have lipid droplets revealed by perilipinl labeling (FIG. 7E). All these results thus show that the adipocytes obtained according to the invention are able to form adipose tissue in vivo.

### CONCLUSION

The differentiation of pluripotent stem cells in two dimensions makes it possible to produce mesodermal progenitors having the ability to differentiate into adipocyte progenitors then into adipocytes when they are subjected to an adipogenic cocktail. The adipocytes obtained by the method according to the invention express the transcription factors characteristic of this cell type and accumulate lipids in triglyceride form. These adipocytes, once transplanted, are able to form panniculus adiposa in vivo. Moreover, the inventors have shown that this method makes it possible to produce beige-type human adipocytes heretofore not described in vitro. Finally, this method makes it possible to produce adipocytes in large quantities in only twenty days from undifferentiated pluripotent stem cells.

### BIBLIOGRAPHICAL REFERENCES

Ahfeldt et al. Nat Cell Biol. 2012 Jan. 15; 14(2):209-19

Chen et al. Nat Methods. 2011 May; 8(5):424-9.

Chung et al. Cell Stem Cell. 2008 Feb. 7; 2(2): 113-7

Gaj et al. Trends in Biotech. Volume 31, Issue 7, July 2013, Pages 397-405

Guenantin et al. Semin Cell Dev Biol. 2014 May; 29C:148-157

Mohammadi et al. Science. 1997; 276:955-960

Patel et al. Stem Cell Rev. September 2010; 6(3): 367-380.

Pittenger et al. Science. 1999 Apr. 2; 284(5411):143-7.

Rossant et al., J Embryol Exp Morphol 1982; 70:90-112

Takahashi, et al. (2007). Cell 131 (5): 861-872

Yamanaka, Cell Stem Cell, 2010. 7(1): p. 1-2.

Yu et al., 2007 Science 318 (5858): 1917-1920

Zuk et al. Tissue Eng, 2001. 7(2): p. 211-28.

Zuk, 2013, ISRN Stem Cells, vol. 2013, Article ID 713959.

Taura et al., FEBS Lett. 2009 Mar. 18; 583(6):1029-33

Xiong et al., Aging (Albany N.Y.). 2013 April; 5(4):288-303

Noguchi et al., Stem Cells Dev. 2013 Nov. 1; 22(21):2895-905

1-18. (canceled)

19. A method for in vitro production of adipocyte progenitors comprising:
culturing pluripotent stem cells on an adherent culture system and in a serum-free culture medium;
contacting said pluripotent stem cells with a mesodermal differentiation medium until obtaining mesodermal progenitors; and
contacting said mesodermal progenitors with an adipogenic differentiation medium until obtaining adipocyte progenitors,
and optionally collecting the adipocyte progenitors thus obtained.

20. The method according to claim 19, wherein the pluripotent stem cells are induced pluripotent stem cells.

21. The method according to claim 19, wherein the mesodermal differentiation medium is a serum-free culture medium comprising one or more morphogens belonging to the TGF-β superfamily, preferably selected from the group consisting of activin A, activin B, BMP-4 protein, BMP-2 protein, TGF-β1, TGF-β2 and TGF-β3, and any combination thereof.

22. The method according to claim 19, wherein the mesodermal differentiation medium is a serum-free culture medium comprising:
(i) a morphogen selected from the group consisting of activin A and activin B; and
(ii) a morphogen selected from the group consisting of BMP-4 protein, BMP-2 protein, TGF-β1, TGF-β2 and TGF-β3, and any combination thereof.

23. The method according to claim 19, wherein the serum-free culture medium is a medium suitable for culturing hematopoietic cells.

24. The method according to claim 19, wherein the pluripotent stem cells are contacted with the mesodermal differentiation medium when the culture reaches about 50% to about 90% confluence.

25. The method according to claim 19, wherein the adipogenic differentiation medium is a culture medium comprising insulin, one of its analogues or IGF-1, a glucocorticoid and an agent that increases intracellular cyclic adenosine monophosphate (cAMP).

26. The method according to claim 19, wherein the adipogenic differentiation medium comprises insulin, dexamethasone and 3-isobutyl-1-methylxanthine.

27. The method according to claim 25, wherein the adipogenic differentiation medium further comprises indomethacin.

28. The method according to claim 26, wherein the adipogenic differentiation medium further comprises indomethacin.

29. A method for in vitro production of adipocytes comprising contacting the adipocyte progenitors obtained by the method according to claim 19 with an adipocyte maturation medium until obtaining adipocytes.

30. The method according to claim 29, wherein the adipocyte maturation medium is a culture medium comprising insulin.

31. A method of treating lipodystrophy comprising the administration of adipocyte progenitors obtained by the method according to claim 19 to a subject in need of treatment.

32. The method according to claim 31, wherein the adipocyte progenitors arise from induced pluripotent stem cells obtained from somatic cells from the subject to be treated.

33. A method of treating lipodystrophy comprising the administration of adipocytes obtained by the method according to claim 29 to a subject in need of treatment.

34. The method according to claim 33, wherein the adipocytes arise from induced pluripotent stem cells obtained from somatic cells from the subject to be treated.

35. A method of treating a glycemic control abnormality or dyslipidemia comprising administering adipocyte progenitors obtained by the method of claim 19 to a subject in need of treatment.

36. The method according to claim 35, wherein the adipocyte progenitors arise from induced pluripotent stem cells obtained from somatic cells from the subject to be treated.

37. A method of treating a glycemic control abnormality or dyslipidemia comprising administering adipocytes obtained by the method of claim 29 to a subject in need of treatment.

38. The method according to claim 37, wherein the adipocytes arise from induced pluripotent stem cells obtained from somatic cells from the subject to be treated.

39. A kit for in vitro production of adipocyte progenitors or adipocytes comprising:
a first container containing one or more morphogens belonging to the TGF-β superfamily;
a second container containing (i) insulin, one of its analogues or IGF-1, (ii) a glucocorticoid and (iii) an agent that increases intracellular cyclic adenosine monophosphate (cAMP); and
optionally a third container containing insulin.

40. The kit according to claim 21, wherein:
the first container contains activin A and/or BMP-4; and
the second container contains insulin, dexamethasone and IBMX, and optionally indomethacin.

41. A method for screening molecules that stimulate the thermogenic activity of adipocytes comprising:
contacting the adipocytes obtained by the method according to claim 11 with one or more candidate molecules; and
selecting the molecules that stimulate the thermogenic activity of the adipocytes.
